SEQUENCE LISTING (11 sequences)

SEQ ID NO: 1 - DNA, 2061 bp, Homo sapiens
gccccaggagaggcagagagtgagggaaagggcctggccggcatgcacagataggatcacggtcctgggagaattcctgctcttatagtctaacctaccatggcttctcttttctcaaggctccctcatgctgccctttggccctagtggctggtttccagggctgaggggactgagtgagctgcctgagaaaagagggtagggaacagaaaagccagccaggagctgtgggaggaaacgccctcagtaaagatgaccgcggtcactgttatctaaacgcaagtgaagccgagtcacaggacccggatgttgtcagttcgacggtaaacgaccctgccagcttccaagagggcggcttcactgtgcgaataggtgagaagccaagaaggaggcgcgctggagttacttccgcccggttctccttcccgcagtctgcagccggagtaagatggcggcgctgagggctttgtgcggcttccggggcgtcgcggcccaggtgctgcggcctggggctggagtccgattgccgattcagcccagcagaggtgttcggcagtggcagccagatgtggaatgggcacagcagtttgggggagctgttatgtacccaagcaaagaaacagcccactggaagcctccaccttggaatgatgtggaccctccaaaggacacaattgtgaagaacattaccctgaactttgggccccaacacccagcagcgcatggtgtcctgcgactagtgatggaattgagtggggagatggtgcggaagtgtgatcctcacatcgggctcctgcaccgaggcactgagaagctcattgaatacaagacctatcttcaggcccttccatactttgaccggctagactatgtgtccatgatgtgtaacgaacaggcctattctctagctgtggagaagttgctaaacatccggcctcctcctcgggcacagtggatccgagtgctgtttggagaaatcacacgtttgttgaaccacatcatggctgtgaccacacatgccctggaccttggggccatgacccctttcttctggctgtttgaagaaagggagaagatgtttgagttctacgagcgagtgtctggagcccgaatgcatgctgcttatatccggccaggaggagtgcaccaggacctaccccttgggcttatggatgacatttatcagttttctaagaacttctctcttcggcttgatgagttggaggagttgctgaccaacaataggatctggcgaaatcggacaattgacattggggttgtaacagcagaagaagcacttaactatggttttagtggagtgatgcttcggggctcaggcatccagtgggacctgcggaagacccagccctatgatgtttacgaccaggttgagtttgatgttcctgttggttctcgaggggactgctatgataggtacctgtgccgggtggaggagatgcgccagtccctgagaattatcgcacagtgtctaaacaagatgcctcctggggagatcaaggttgatgatgccaaagtgtctccacctaagcgagcagagatgaagacttccatggagtcactgattcatcactttaagttgtatactgagggctaccaagttcctccaggagccacatatactgccattgaggctcccaagggagagtttggggtgtacctggtgtctgatggcagcagccgcccttatcgatgcaagatcaaggctcctggttttgcccatctggctggtttggacaagatgtctaagggacacatgttggcagatgtcgttgccatcataggtacccaagatattgtatttggagaagtagatcggtgagcaggggagcagcgtttgatcccccctgcctatcagcttcttctgtggagcctgttcctcactggaaattggcctctgtgtgtgtgtgtgtgtgtgtgtgtgtgtgtgtatgttcgtgtacacttggctgtcaggctttctgtgcatgtactaaaaaaggagaaattataataaattagccgtcttgcggcccctaggcctaaaaaaaaaaaaaaaaaaaa

SEQ ID NO: 2 - DNA, 1847 bp, Homo sapiens
agtctgcagccggagtaagatggcggcgctgagggctttgtgcggcttccggggcgtcgcggcccaggtgctgcggcctggggctggagtccgattgccgattcagcccagcagaggtgttcggcagtggcagccagatgtggaatgggcacagcagtttgggggagctgttatgtacccaagcaaagaaacagcccactggaagcctccaccttggaatgatgtggaccctccaaaggacacaattgtgaagaacattaccctgaactttgggccccaacacccagcagcgcatggtgtcctgcgactagtgatggaattgagtggggagatggtgcggaagtgtgatcctcacatcgggctcctgcaccgaggcactgagaagctcattgaatacaagacctatcttcaggcccttccatactttgaccggctagactatgtgtccatgatgtgtaacgaacaggcctattctctagctgtggagaagttgctaaacatccggcctcctcctcgggcacagtggatccgagtgctgtttggagaaatcacacgtttgttgaaccacatcatggctgtgaccacacatgccctggaccttggggccatgacccctttcttctggctgtttgaagaaagggagaagatgtttgagttctacgagcgagtgtctggagcccgaatgcatgctgcttatatccggccaggaggagtgcaccaggacctaccccttgggcttatggatgacatttatcagttttctaagaacttctctcttcggcttgatgagttggaggagttgctgaccaacaataggatctggcgaaatcggacaattgacattggggttgtaacagcagaagaagcacttaactatggttttagtggagtgatgcttcggggctcaggcatccagtgggacctgcggaagacccagccctatgatgtttacgaccaggttgagtttgatgttcctgttggttctcgaggggactgctatgataggtacctgtgccgggtggaggagatgcgccagtccctgagaattatcgcacagtgtctaaacaagatgcctcctggggagatcaaggttgatgatgccaaagtgtctccacctaagcgagcagagatgaagacttccatggagtcactgattcatcactttaagttgtatactgagggctaccaagttcctccaggagccacatatactgccattgaggctcccaagggagagtttggggtgtacctggtgtctgatggcagcagccgcccttatcgatgcaagatcaaggctcctggttttgcccatctggctggtttggacaagatgtctaagggacacatgttggcagatgtcgttgccatcataggtacgaggcctattgtgtagtagaggtatcctagacaaaggagttcgggacgcccactggggacagaaggagaacacttcctgttcaccataggccatggcatggactcgggtcctcaatcttttgagcacagtaatgggttctggatcttgggtaacaccactttttttgtttgttttgcctcacaacaggaagataagtaacatcacttttttcctccatcctctcacctaggtacccaagatattgtatttggagaagtagatcggtgagcaggggagcagcgtttgatcccccctgcctatcagcttcttctgtggagcctgttcctcactggaaattggcctctgtgtgtgtgtgtgtgtgtgtgtgtgtgtgtatgttcatgtacacttggctgtcaggctttctgtgcatgtactaaaaaaggagaaattataataaattagccgtcttgcggcccctaggcctaaa

SEQ ID NO: 3 - protein, 463 aa, Homo sapiens
MAALRALCGFRGVAAQVLRPGAGVRLPIQPSRGVRQWQPDVEWAQQFGGAVMYPSKETAHWKPPPWNDVDPPKDTIVKNITLNFGPQHPAAHGVLRLVMELSGEMVRKCDPHIGLLHRGTEKLIEYKTYLQALPYFDRLDYVSMMCNEQAYSLAVEKLLNIRPPPRAQWIRVLFGEITRLLNHIMAVTTHALDLGAMTPFFWLFEEREKMFEFYERVSGARMHAAYIRPGGVHQDLPLGLMDDIYQFSKNFSLRLDELEELLTNNRIWRNRTIDIGVVTAEEALNYGFSGVMLRGSGIQWDLRKTQPYDVYDQVEFDVPVGSRGDCYDRYLCRVEEMRQSLRIIAQCLNKMPPGEIKVDDAKVSPPKRAEMKTSMESLIHHFKLYTEGYQVPPGATYTAIEAPKGEFGVYLVSDGSSRPYRCKIKAPGFAHLAGLDKMSKGHMLADVVAIIGTQDIVFGEVDR

SEQ ID NO: 4 - DNA, 20 bp, Artificial Sequence
  Synthetic Construct
acgtttgttgaaccacatca

SEQ ID NO: 5 - DNA, 20 bp, Artificial Sequence
  Synthetic Construct
cagtggatccgagtgctgtt

SEQ ID NO: 6 - DNA, 25 bp, Artificial Sequence
  Synthetic Construct
caccgcagtggatccgagtgctgtt

SEQ ID NO: 7 - DNA, 25 bp, Artificial Sequence
  Synthetic Construct
aaacaacagcactcggatccactgc

SEQ ID NO: 8 - DNA, 25 bp, Artificial Sequence
  Synthetic Construct
caccgacgtttgttgaaccacatca

SEQ ID NO: 9 - DNA, 25 bp, Artificial Sequence
  Synthetic Construct
aaactgatgtggttcaacaaacgtc

SEQ ID NO: 10 - DNA, 23 bp, Artificial Sequence
  Synthetic Construct
gagggcctatttcccatgattcc

SEQ ID NO: 11 - RNA, 2061 nt, Homo sapiens
gccccaggagaggcagagagugagggaaagggccuggccggcaugcacagauaggaucacgguccugggagaauuccugcucuuauagucuaaccuaccauggcuucucuuuucucaaggcucccucaugcugcccuuuggcccuaguggcugguuuccagggcugaggggacugagugagcugccugagaaaagaggguagggaacagaaaagccagccaggagcugugggaggaaacgcccucaguaaagaugaccgcggucacuguuaucuaaacgcaagugaagccgagucacaggacccggauguugucaguucgacgguaaacgacccugccagcuuccaagagggcggcuucacugugcgaauaggugagaagccaagaaggaggcgcgcuggaguuacuuccgcccgguucuccuucccgcagucugcagccggaguaagauggcggcgcugagggcuuugugcggcuuccggggcgucgcggcccaggugcugcggccuggggcuggaguccgauugccgauucagcccagcagagguguucggcaguggcagccagauguggaaugggcacagcaguuugggggagcuguuauguacccaagcaaagaaacagcccacuggaagccuccaccuuggaaugauguggacccuccaaaggacacaauugugaagaacauuacccugaacuuugggccccaacacccagcagcgcaugguguccugcgacuagugauggaauugaguggggagauggugcggaagugugauccucacaucgggcuccugcaccgaggcacugagaagcucauugaauacaagaccuaucuucaggcccuuccauacuuugaccggcuagacuauguguccaugauguguaacgaacaggccuauucucuagcuguggagaaguugcuaaacauccggccuccuccucgggcacaguggauccgagugcuguuuggagaaaucacacguuuguugaaccacaucauggcugugaccacacaugcccuggaccuuggggccaugaccccuuucuucuggcuguuugaagaaagggagaagauguuugaguucuacgagcgagugucuggagcccgaaugcaugcugcuuauauccggccaggaggagugcaccaggaccuaccccuugggcuuauggaugacauuuaucaguuuucuaagaacuucucucuucggcuugaugaguuggaggaguugcugaccaacaauaggaucuggcgaaaucggacaauugacauugggguuguaacagcagaagaagcacuuaacuaugguuuuaguggagugaugcuucggggcucaggcauccagugggaccugcggaagacccagcccuaugauguuuacgaccagguugaguuugauguuccuguugguucucgaggggacugcuaugauagguaccugugccggguggaggagaugcgccagucccugagaauuaucgcacagugucuaaacaagaugccuccuggggagaucaagguugaugaugccaaagugucuccaccuaagcgagcagagaugaagacuuccauggagucacugauucaucacuuuaaguuguauacugagggcuaccaaguuccuccaggagccacauauacugccauugaggcucccaagggagaguuugggguguaccuggugucugauggcagcagccgcccuuaucgaugcaagaucaaggcuccugguuuugcccaucuggcugguuuggacaagaugucuaagggacacauguuggcagaugucguugccaucauagguacccaagauauuguauuuggagaaguagaucggugagcaggggagcagcguuugauccccccugccuaucagcuucuucuguggagccuguuccucacuggaaauuggccucuguguguguguguguguguguguguguguguguauguucguguacacuuggcugucaggcuuucugugcauguacuaaaaaaggagaaauuauaauaaauuagccgucuugcggccccuaggccuaaaaaaaaaaaaaaaaaaaa

(19) United States
(12) Patent Application Publication
(10) Pub. No.: US 2022/0299500 A1
(43) Pub. Date: Sep. 22, 2022

(54) NDUFS2 SUBUNIT KNOCKOUT HUMAN CELL LINE

(71) Applicant: Virginia Tech Intellectual Properties Inc., Blacksburg, VA (US)

(72) Inventors: Aloka B. BANDARA, Christiansburg, VA (US); David A. BROWN, Blacksburg, VA (US)

(21) Appl. No.: 17/597,995

(22) PCT Filed: Sep. 30, 2020
(86) PCT No.: PCT/US2020/053511

§ 371 Date: Feb. 1, 2022

Related U.S. Application Data
(60) Provisional application No. 62/908,849, filed on Oct. 1, 2019.

(51) Int. Cl. G01N 33/50; C12N 9/02; C12N 15/11; C12N 9/22

(52) U.S. Cl. CPC G01N 33/5079; C12N 9/0036; C12N 15/111; C12N 9/22; C12Y 106/05003; C12N 5/0602

(57) ABSTRACT

Disclosed herein is a recombinant human cell line that does not express functional endogenous NADH dehydrogenase [ubiquinone] iron-sulfur protein 2 (NDUFS2). This knockout cell line can be used to advance the basic understanding of the NDUFS2 protein subunit in overall complex I assembly and function. Furthermore, this cell line can be used to test therapeutic agents to fix or bypass the dysfunctional complex I.

### CROSS-REFERENCE TO RELATED APPLICATIONS

This application claims benefit of U.S. Provisional Application No. 62/908,849, filed Oct. 1, 2019, which is hereby incorporated herein by reference in its entirety.

### SEQUENCE LISTING

This application contains a sequence listing filed in electronic form as an ASCII.txt file entitled “222204_2300_Sequence_Listing_ST25” created on Sep. 30, 2020. The content of the sequence listing is incorporated herein in its entirety.

### BACKGROUND

Mitochondria are organelles that function as cellular powerhouses, generating >90% of the body's energy (adenosine triphosphate; ATP). Mitochondrial dysfunction leads to diseases in organs or tissues that have high energy demands, such as heart, muscle, brain, eye muscles, liver, and kidney. ATP synthesis in mitochondria occurs as a result of flow of electron through the electron transport chain (ETC). The ETC is comprised of four protein complexes (complex I through IV). Complex I is the largest and most intricate of the enzyme complexes of the ETC, and is most commonly impaired in disease states. Defects in complex I leads to several different clinical pathologies that include (but are not limited to) hepatopathy, cardiomyopathy, muscle myopathies, fatal congenital lactic acidosis, Leber's hereditary optic neuropathy, and Leigh syndrome.

### SUMMARY

NADH:ubiquinone oxidoreductase (complex I; EC 1.6.5.3) is the first multimeric complex of the ETC and catalyzes the first step of the ETC in the mitochondrial oxidative phosphorylation (OXPHOS) system. Complex I is responsible for transferring electrons from nicotinamide adenine dinucleotide (NADH) to ubiquinone via flavin mononucleotide (FMN) and iron-sulfur clusters. The transfer of every two electrons from NADH to ubiquinone is coupled to the transfer of four protons across the mitochondrial inner-membrane (Carroll J, et al. Mol Cell Proteomics 2003, 2(2):117-126; Sazanov L A, et al. Biochemistry 2000, 39(24):7229-7235). Structurally, the 1 MDa size complex I is the largest and most intricate of the enzyme complexes of the ETC. The mammalian complex I is assembled from at least 45 individual subunits of which seven subunits are mitochondrially-encoded while the rest of 38 subunits are nuclear-encoded (Carroll J, et al. J Biol Chem 2006, 281(43):32724-32727).

Mammalian complex I is an L-shaped structure of which, its hydrophilic peripheral arm protrudes into the matrix of the organelle while its hydrophobic orthogonal arm is embedded in the lipid bilayer of the inner mitochondrial membrane (Grigorieff N. J Mol Biol 1998, 277(5):1033-1046). The interface region between the two arms is believed to be critical for the function of complex I enzyme as this (interface) region constitutes a large quinone or inhibitor binding pocket (Efremov R G, et al. Nature 2010, 465(7297):441-445; Hunte C, et al. Science 2010, 329(5990):448-451). The interface region is comprised of three major protein subunits, namely, NADH dehydrogenase [ubiquinone] iron-sulfur protein 2 (NDUFS2 or 49 kDa), NADH dehydrogenase [ubiquinone] iron-sulfur protein 7 (NDUFS7 or PSST), and NADH dehydrogenase 1 (ND1). Inhibitors in particular bind to the interfacial region between the peripheral and membrane arms. The large cleft formed by the NDUFS2 and NDUFS7 subunits is the site where ubiquinone is reduced by electrons from the terminal Fe—S cluster (Tocilescu M A, et al. J Biol Chem 2007, 282(40):29514-29520; Tocilescu M A, et al. Biochim Biophys Acta 2010, 1797(12):1883-1890). Therefore, the interfacial juncture between the peripheral and membrane domains appears to be a “critical region” or a “hot spot” for the binding of inhibitors and ubiquinone. Nevertheless, the knowledge on organizational or functional aspects of the interfacial juncture of complex I is still scarce. Disclosed herein is the importance of NDUFS2 for in vitro growth, cell-membrane integrity, ATP synthesis, oxygen consumption, and complex I respiration of a human cell line.

Complex I is comprised of at least 45 protein subunits. NADH dehydrogenase [ubiquinone] iron-sulfur protein 2 (NDUFS2) is one of the subunits of complex I. Mutations in this specific subunit are noted in several pathologies, yet effective preclinical models of this knockout are lacking. Using a CRISPR/cas9 approach, a NDUFS2-deficient mutant was constructed in a human embryonic kidney cell line, HEK293. The resultant cell line had a clear decrement in the expression of NSUDS2 protein.

Unlike the control HEK293 cell line, the constructed mutant cell line (designated HEK293ΔNDUFS2) grew slower, produced less ATP, possessed weaker cell membranes, and displayed reduced complex I respiration. This knockout cell line can be used to advance the basic understanding of the NDUFS2 protein subunit in overall complex I assembly and function. Furthermore, this cell line can be used to test therapeutic agents to fix or bypass the dysfunctional complex I.

Disclosed herein is a recombinant human cell line, wherein each cell of the recombinant human cell line has an inactivation mutation in a NADH dehydrogenase [ubiquinone] iron-sulfur protein 2 (NDUFS2) gene, wherein the human cell line does not express a functional NDUFS2 protein. In some embodiments, the inactivation mutation involves a deletion or non-sense mutation in at least one coding region of the NDUFS2 gene. In some embodiments, a cDNA for the NDUFS2 gene has the nucleotide sequence SEQ ID NO:1.

In some embodiments, the NDUFS2 gene has been mutated or deleted using CRISPR/Cas9 genome editing with a short guide RNAs (sgRNA) that targets a coding region in the NDUFS2 gene. For example, in some embodiments, the sgRNA comprises the nucleic acid sequence SEQ ID NO:6, 7, 8, or 9.

In some embodiments, the cell is a human embryonic kidney (HEK) cell, such as a HEK293 cell. Similar mutants can be generated in other eukaryotic cell lines, including mouse cardiomyocyte HL-1.

In some embodiments, the cell is stably or transiently transfected with a first heterologous nucleic acid sequence. In some embodiments, the cell is stably or transiently transfected with a second heterologous nucleic acid sequence.

In some embodiments, the cells are contained in a culture or growth medium, or in a medium suitable for long-term storage.

Also disclosed herein is a screening method that involves contacting the recombinant human cell line disclosed herein with a candidate agent, and assaying the cell line for ATP synthesis, cell growth, complex I respiration, or a combination thereof, wherein an increase in any one of ATP synthesis, cell growth, or complex I respiration is an indication that the candidate agent may be useful for treating a subject with a complex I defect. In some embodiments, the complex I defect comprises hepatopathy, cardiomyopathy, muscle myopathies, fatal congenital lactic acidosis, Leber's hereditary optic neuropathy, or Leigh syndrome.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the gene sequence of NDUFS2 (SEQ ID NO:1) with CRISPR targeting sites. The sgRNA and PAM sequences predicted by the online CRISPOR program are depicted; the specific site for mutation (at 970-bp) is shown with the first arrow. The sgRNA and PAM sequences predicted by the online idtdna program are depicted; the specific site for mutation (at 1002-bp) is shown with the second arrow.

FIG. 2 shows expression of NDUFS2 protein. The protein extracted from cells were suspended in Laemmli Sample Buffer, boiled, and run in 4-20% Mini-PROTEAN protein gels. Proteins from gels were transferred to PVDF Membrane, and the membranes were blocked, incubated with rabbit polyclonal antibodies to NDUFS2 (left) or rabbit polyclonal antibodies to β-Actin (right), and incubated with the secondary antibody IRDye® 800CW Donkey anti-Rabbit IgG (H+L). Lanes 1: protein marker (Precision Plus Protein™ Kaleidoscope™ Standards; BioRad); 2: protein of strain HEK293; and 3: protein of mutant HEK293ΔNDUFS2.

FIG. 3 shows cellular respiration measured using Oroboros Oxygraph 2k. Oxygen consumption rates (OCR) measured per million cells in response to treatment with glutamate-malate (G/M), ADP, and FCCP are shown. The mean OCR values were compared between the parent and the mutant. The p values for the differences between the mean values were <0.0001, 0.001, 0.747, and 0.645 respectively for complex I respiration, complex II respiration, OXPHOS capacity, and maximal respiration. The mean values significantly different between the parent and the mutant are indicated by*. Error bars represent the SE of the mean.

FIG. 4 shows ATP synthesis of strains measured by using the Mitochondral ToxGlo Assay. The ATP synthesis capability of strains at the absence of the respiratory inhibitors and at the presence of the complex III respiratory inhibitor Antimycin A in reaction medium is shown. The measurements are expressed as total luminescence per 10,000 cells. The mean luminescence values were compared between the parent and the mutant. The p values for the differences between the mean values were 0.0005, 0.9266, 0.8731, 0.1264 0.6199, 0.3563, 0.0014, and 0.0067 respectively for 0, −2.7. −2.5, −2.3, −2.2, −2.0, −1.8, and −1.6 Log [Antimycin] g/L. The mean values significantly different between the parent and the mutant are indicated by *. Error bars represent the SE of the mean.

FIG. 5 shows cell-membrane porousness measured by using the Mitochondrial ToxGlo Assay. The recorded fluorescence was equivalent to the amount of substrate passaged into the cell via cell membrane. The greater the porousness of the cell membrane the higher the fluorescence recorded. The membrane porousness of strains at the absence of the respiratory inhibitors and at the presence of the complex III respiratory inhibitor Antimycin A in reaction medium is shown. The measurements are expressed as total fluorescence per 10,000 cells. The p values for the differences between the mean values were 0.0221, 0.0409, 0.0013, 0.0103, 0.8506, and 0.0011 respectively for 0, −2.3, −2.2, −2.0, −1.8, and −1.6 Log [Antimycin] g/L. The mean values significantly different between the parent and the mutant are indicated by *. Error bars represent the SE of the mean.

FIG. 6 shows distribution of mitochondria examined by confocal microscopy. Micrographs of the parent strain HEK293 (top panel) and the mutant HEK293ΔNDUFS2 (bottom panel) grown for 48 hours and subsequently treated with MitoTracker Green FM are shown.

FIG. 7 shows in vitro growth of strains in culture media. Aliquots of 50,000 cells of parent strain HEK293 or the mutant HEK293ΔNDUFS2 were introduced into 25 cm² flasks carrying 5 ml growth media. Cultures were incubated for 4 days, cells harvested by trypsin-EDTA treatment, cell numbers quantified in triplicates, and doubling time was calculated between the day 0 and day 4. The p value for the difference between the mean values (indicated by *) was 0.0477. Error bars represent the SE of the mean.

FIG. 8 shows ATP synthesis of the mutant grown in culture media supplemented with drugs, measured using the Mitochondral ToxGlo Assay. Aliquots of 2000 cells of the mutant HEK293ΔNDUFS2 were introduced into each well on 96-well plate in growth media supplemented with 1 μM of SBT-222, SBT-220, elamipretide, or idebenone. Fresh media supplemented with therapeutics were added on days 0, 3 and 5. ATP synthesis capability of the strain six days after treatment is shown. The measurements are expressed as luminescence normalized to the values of DMSO control. The mean values of each drug treatment group was compared individually with that of the DMSO treated group by unpaired t test. The p values for the drugs SBT-222, SBT-220, elamiprertide, and idebenone respectively were 0.0014, 0.0266, 0.0056, and 0.0101. Statistically significant differences are shown with *. Error bars represent the SE of the mean.

FIGS. 9A to 9D show respiration, glycolysis, and ATP synthesis of cell lines. FIG. 9A shows oxygen consumption rates measured using Oroboros O2k per million saponin-permiabelized cells in response to treatment with glutamate-malate (G/M), succinate, ADP, and FCCP. The mean oxygen consumption rates were compared between the parent and the mutant. The p values for the differences between the mean values were <0.0001, 0.001, 0.747, and 0.645 respectively for complex I respiration, complex II respiration, OXPHOS capacity, and maximal respiration. FIG. 9B shows ATP synthesis of cells measured using Mitochondrial ToxGlo Assay. The measurements are expressed as total luminescence per 10,000 cells. The mean luminescence values were compared between the parent and the mutant. The p value for the differences between the mean values was <0.0001. FIGS. 9C and 9D show extracellular acidification rate (ECAR) measured using Agilent Seahorse XFe96 analyzer when 10,000 live cells/well (FIG. 9C) or 20,000 live cells/well (FIG. 9D) used in assays. The mean ECAR values were compared between the parent and the mutant. The p values for the differences between the mean values in response to injections of assay medium, glucose, ATPase inhibitor oligomycin, and 2-deoxy glucose (2-DG) respectively were 0.0342, <0.0001, <0.0001, and 0.6332 at 10,000 cells/well and 0.0358, <0.0001, <0.0001, and 0.9541 at 20,000 cells/well. The ECAR measurements are expressed in mpH/min. The mean values significantly different between the parent and the mutant are indicated by *. Error bars represent the SE of the mean.

FIGS. 10A to 10C show growth and membrane porousness of cell lines. The number of cells harvested after four and six days of culture (FIG. 10A), doubling times (FIG. 10B), and membrane porousness (FIG. 10C) of parent and mutant cell lines are shown. The p values for the differences between the means of two cell lines were 0.019 for cell numbers at day 4, 0.027 for cell numbers at day 6, 0.0477 for doubling time, and 0.0221 for membrane porousness. The mean values significantly different between cell lines are indicated by *. Error bars represent the SE of the mean.

FIGS. 11A to 11C show restoration of the mutant defects with use of mitochondrial therapeutic idebenone. FIG. 11A shows the oxygen consumption rates per million permeabilized cells treated with DMSO or idebenone. The p values for the differences between the mean values of DMSO control versus idebenone treatment were <0.110, 0.024, 0.0001, 0.013, and 0.0066 respectively for complex I respiration, respiration after DMSO/idebenone treatment, complex II respiration, OXPHOS capacity, and maximal respiration. FIG. 11B shows ATP pools of cells treated with idebenone for six days. The measurements are expressed as luminescence normalized to the values of DMSO control. The p value for the difference between DMSO control and idebenone treatement was 0.0101. FIG. 11C shows the number of cells recovered from the mutant cultures treated with 0.5, 1, or 5 μM idebenone or DMSO control for 10 days. The p value for the differences in cell numbers between DMSO control versus treatment with idebenone at 0.5, 1, and 5 μM were 0.0011, 0.9577, and 0.99539, respectively. The mean values significantly different between the DMSO control idebenone treatments are indicted by *. Error bars represent the SE of the mean.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “cell” is used in its usual biological sense, and does not refer to an entire multicellular organism. The cell can, for example, be in vitro, e.g., in cell culture.

The term “host cell” includes an individual cell or cell culture which can be or has been a recipient of any virus or recombinant vector(s). Host cells include progeny of a single host cell, and the progeny may not necessarily be completely identical (in morphology or in total DNA complement) to the original parent cell due to natural, accidental, or deliberate mutation and/or change. A host cell includes cells infected or transfected with a virus or recombinant vector.

The cells of disclosed herein are grown in any vessel, flask, tissue culture dish or device used for culturing cells that provides a suitable surface for cell attachment and spreading (e.g., Culture of Hematopoietic Cells (Culture of Specialized Cells) R. I. Freshney et al., Ed., I. Freshney; Wiley-Liss 1994; incorporated by reference herein). As used herein, the term “attachment” refers to cell adherence and spreading on a surface in such a device, where factors promoting cell attachment and spreading directly contact the cultured cells, Cell growth is maintained directly on surfaces of the culture vessel or on supplemental inserts such as cartridges or membranes placed within the vessel, Appropriate attachment and spreading surfaces are produced either by initially selecting a suitable surface material or by subsequently treating an existing surface. Common treatments are well known and include coating surfaces with compositions that promote attachment and spreading. Such compositions are also well known and include polybasic amino acids such as polyomithine and polylysine. Furthermore, the attachment surface may be coated or provided with a known extracellular matrix protein or with compositions or artificial environments that are functionally equivalent to an in vivo extracellular matrix. Typical cell matrix compositions are well known and include laminin, collagen and fibronectin. Other extra cellular matrix proteins or artificial extracellular matrix environments, which mimic an in vivo extracellular matrix, are known in the art (see e.g., Synthetic Biodegradable Polymer Scaffolds (Tissue Engineering) A. Atala and D. J. Mooney (Eds.) Birkhauser, 1997).

The term “cell line” refers to a population or mixture of cells of common origin growing together after several passages in vitro. By growing together in the same medium and culture conditions, the cells of the cell line share the characteristics of generally similar growth rates, temperature, gas phase, nutritional and surface requirements.

Clonal cells are those which are descended from a single cell. As a practical matter, it is difficult to obtain pure cloned cell cultures of mammalian cells. A high degree of cell purity can be obtained by successive rounds of cell enrichment. As used herein, a cell culture in which at least 90% of the cells possess a defined set of traits is termed a cloned cell culture.

The term “knockout” as used herein refers to any loss of function mutation in a target gene sequence, including complete or partial removal of the target gene sequence, or incorporation of a missense or nonsense mutation that results in translation of a non-functional protein.

### Genetic Mutation

In some embodiments, the mutation of the NDUFS2 gene is any mutation that creates an inactive NDUFS2 protein. In some embodiments, the NDUFS2 protein has the amino acid sequence:

MAALRALCGFRGVAAQVLRPGAGVRLPIQPSRGVRQWQPDVEWAQQFGGA
VMYPSKETAHWKPPPWNDVDPPKDTIVKNITLNFGPQHPAAHGVLRLVME
LSGEMVRKCDPHIGLLHRGTEKLIEYKTYLQALPYFDRLDYVSMMCNEQA
YSLAVEKLLNIRPPPRAQWIRVLFGEITRLLNHIMAVTTHALDLGAMTPF
FWLFEEREKMFEFYERVSGARMHAAYIRPGGVHQDLPLGLMDDIYQFSKN
FSLRLDELEELLTNNRIWRNRTIDIGVVTAEEALNYGFSGVMLRGSGIQW
DLRKTQPYDVYDQVEFDVPVGSRGDCYDRYLCRVEEMRQSLRIIAQCLNK
MPPGElKVDDAKVSPPKRAEMKTSMESLIHHFKLYTEGYQVPPGATYTAI
EAPKGEFGVYLVSDGSSRPYRCKIKAPGFAHLAGLDKMSKGHMLADVVAI
IGTQDIVFGEVDR(SEQ ID NO: 3, EC 1.6.5.3).

The cDNA sequence for human NDUFS2 is provided below as SEQ ID NO:1 (BC008868):

(SEQ ID NO: 1)
gccccaggagaggcagagagtgagggaaagggcctggccggcatgcacag
ataggatcacggtcctgggagaattcctgctcttatagtctaacctacca
tggcttctcttttctcaaggctccctcatgctgccctttggccctagtgg
ctggtttccagggctgaggggactgagtgagctgcctgagaaaagagggt
agggaacagaaaagccagccaggagctgtgggaggaaacgccctcagtaa
agatgaccgcggtcactgttatctaaacgcaagtgaagccgagtcacagg
acccggatgttgtcagttcgacggtaaacgaccctgccagcttccaagag
ggcggcttcactgtgcgaataggtgagaagccaagaaggaggcgcgctgg
agttacttccgcccggttctccttcccgcagtctgcagccggagtaagat
ggcggcgctgagggctttgtgcggcttccggggcgtcgcggcccaggtgc
tgcggcctggggctggagtccgattgccgattcagcccagcagaggtgtt
cggcagtggcagccagatgtggaatgggcacagcagtttgggggagctgt
tatgtacccaagcaaagaaacagcccactggaagcctccaccttggaatg
atgtggaccctccaaaggacacaattgtgaagaacattaccctgaacttt
gggccccaacacccagcagcgcatggtgtcctgcgactagtgatggaatt
gagtggggagatggtgcggaagtgtgatcctcacatcgggctcctgcacc
gaggcactgagaagctcattgaatacaagacctatcttcaggcccttcca
tactttgaccggctagactatgtgtccatgatgtgtaacgaacaggccta
ttctctagctgtggagaagttgctaaacatccggcctcctcctcgggcac
agtggatccgagtgctgtttggagaaatcacacgtttgttgaaccacatc
atggctgtgaccacacatgccctggaccttggggccatgacccctttctt
ctggctgtttgaagaaagggagaagatgtttgagttctacgagcgagtgt
ctggagcccgaatgcatgctgcttatatccggccaggaggagtgcaccag
gacctaccccttgggcttatggatgacatttatcagttttctaagaactt
ctctcttcggcttgatgagttggaggagttgctgaccaacaataggatct
ggcgaaatcggacaattgacattggggttgtaacagcagaagaagcactt
aactatggttttagtggagtgatgcttcggggctcaggcatccagtggga
cctgcggaagacccagccctatgatgtttacgaccaggttgagtttgatg
ttcctgttggttctcgaggggactgctatgataggtacctgtgccgggtg
gaggagatgcgccagtccctgagaattatcgcacagtgtctaaacaagat
gcctcctggggagatcaaggttgatgatgccaaagtgtctccacctaagc
gagcagagatgaagacttccatggagtcactgattcatcactttaagttg
tatactgagggctaccaagttcctccaggagccacatatactgccattga
ggctcccaagggagagtttggggtgtacctggtgtctgatggcagcagcc
gcccttatcgatgcaagatcaaggctcctggttttgcccatctggctggt
ttggacaagatgtctaagggacacatgttggcagatgtcgttgccatcat
aggtacccaagatattgtatttggagaagtagatcggtgagcaggggagc
agcgtttgatcccccctgcctatcagcttcttctgtggagcctgttcctc
actggaaattggcctctgtgtgtgtgtgtgtgtgtgtgtgtgtgtgtgta
tgttcgtgtacacttggctgtcaggctttctgtgcatgtactaaaaaagg
agaaattataataaattagccgtcttgcggcccctaggcctaaaaaaaaa
aaaaaaaaaaa.

Therefore, an mRNA sequence for human NDUFS2 is provided below as SEQ ID NO:11:

(SEQ ID NO: 11)
gccccaggagaggcagagagugagggaaagggccuggccggcaugcacag
auaggaucacgguccugggagaauuccugcucuuauagucuaaccuacca
uggcuucucuuuucucaaggcucccucaugcugcccuuuggccuaguggc
ugguuuccagggcugaggggacugagugagcugccugagaaaagagggua
gggaacagaaaagccagccaggagcugugggaggaaacgcccucaguaaa
gaugaccgcggucacuguuaucuaaacgcaagugaagccgagucacagga
cccggauguugucaguucgacgguaaacgacccugccagcuuccaagagg
gcggcuucacugugcgaauaggugagaagccaagaaggaggcgcgcugga
guuacuuccgcccgguucuccuucccgcagucugcagccggaguaagaug
gcggcgcugagggcuuugugcggcuuccggggcgucgcggcccaggugcu
gcggccuggggcuggaguccgauugccgauucagcccagcagagguguuc
ggcaguggcagccagauguggaaugggcacagcaguuugggggagcuguu
auguacccaagcaaagaaacagcccacuggaagccuccaccuuggaauga
uguggacccuccaaaggacacaauugugaagaacauuacccugaacuuug
ggccccaacacccagcagcgcaugguguccugcgacuagugauggaauug
aguggggagauggugcggaagugugauccucacaucgggcuccugcaccg
aggcacugagaagcucauugaauacaagaccuaucuucaggcccuuccau
acuuugaccggcuagacuauguguccaugauguguaacgaacaggccuau
ucucuagcuguggagaaguugcuaaacauccggccuccuccucgggcaca
guggauccgagugcuguuuggagaaaucacacguuuguugaaccacauca
uggcugugaccacacaugcccuggaccuuggggccaugaccccuuucuuc
uggcuguuugaagaaagggagaagauguuugaguucuacgagcgaguguc
uggagcccgaaugcaugcugcuuauauccggccaggaggagugcaccagg
accuaccccuugggcuuauggaugacauuuaucaguuuucuaagaacuuc
ucucuucggcuugaugaguuggaggaguugcugaccaacaauaggaucug
gcgaaaucggacaauugacauugggguuguaacagcagaagaagcacuua
acuaugguuuuaguggagugaugcuucggggcucaggcauccagugggac
cugcggaagacccagcccuaugauguuuacgaccagguugaguuugaugu
uccuguugguucucgaggggacugcuaugauagguaccugugccgggugg
aggagaugcgccagucccugagaauuaucgcacagugucuaaacaagaug
ccuccuggggagaucaagguugaugaugccaaagugucuccaccuaagcg
agcagagaugaagacuuccauggagucacugauucaucacuuuaaguugu
auacugagggcuaccaaguuccuccaggagccacauauacugccauugag
gcucccaagggagaguuugggguguaccuggugucugauggcagcagccg
cccuuaucgaugcaagaucaaggcuccugguuuugcccaucuggcugguu
uggacaagaugucuaagggacacauguuggcagaugucguugccaucaua
gguacccaagauauuguauuuggagaaguagaucggugagcaggggagca
gcguuugauccccccugccuaucagcuucuucuguggagccuguuccuca
cuggaaauuggccucuguguguguguguguguguguguguguguguguau
guucguguacacuuggcugucaggcuuucugugcauguacuaaaaaagga
gaaauuauaauaaauuagccgucuugcggccccuaggccuaaaaaaaaaa
aaaaaaaaaa.

In some embodiments, the inactivation mutation can occur in any location that will produce a non-functional protein. In some embodiments, the mutation is in a coding region of the NDUFS2 gene, such as nucleotides 449-1840 of SEQ ID NO:1, reproduced in SEQ ID NO:2 (NM_001166159) below:

(SEQ ID NO: 2)
agtctgcagccggagtaagatggcggcgctgagggctttgtgcggcttcc
ggggcgtcgcggcccaggtgctgcggcctggggctggagtccgattgccg
attcagcccagcagaggtgttcggcagtggcagccagatgtggaatgggc
acagcagtttgggggagctgttatgtacccaagcaaagaaacagcccact
ggaagcctccaccttggaatgatgtggaccctccaaaggacacaattgtg
aagaacattaccctgaactttgggccccaacacccagcagcgcatggtgt
cctgcgactagtgatggaattgagtggggagatggtgcggaagtgtgatc
ctcacatcgggctcctgcaccgaggcactgagaagctcattgaatacaag
acctatcttcaggcccttccatactttgaccggctagactatgtgtccat
gatgtgtaacgaacaggcctattctctagctgtggagaagttgctaaaca
tccggcctcctcctcgggcacagtggatccgagtgctgtttggagaaatc
acacgtttgttgaaccacatcatggctgtgaccacacatgccctggacct
tggggccatgacccctttcttctggctgtttgaagaaagggagaagatgt
ttgagttctacgagcgagtgtctggagcccgaatgcatgctgcttatatc
cggccaggaggagtgcaccaggacctaccccttgggcttatggatgacat
ttatcagttttctaagaacttctctcttcggcttgatgagttggaggagt
tgctgaccaacaataggatctggcgaaatcggacaattgacattggggtt
gtaacagcagaagaagcacttaactatggttttagtggagtgatgcttcg
gggctcaggcatccagtgggacctgcggaagacccagccctatgatgttt
acgaccaggttgagtttgatgttcctgttggttctcgaggggactgctat
gataggtacctgtgccgggtggaggagatgcgccagtccctgagaattat
cgcacagtgtctaaacaagatgcctcctggggagatcaaggttgatgatg
ccaaagtgtctccacctaagcgagcagagatgaagacttccatggagtca
ctgattcatcactttaagttgtatactgagggctaccaagttcctccagg
agccacatatactgccattgaggctcccaagggagagtttggggtgtacc
tggtgtctgatggcagcagccgcccttatcgatgcaagatcaaggctcct
ggttttgcccatctggctggtttggacaagatgtctaagggacacatgtt
ggcagatgtcgttgccatcataggtacgaggcctattgtgtagtagaggt
atcctagacaaaggagttcgggacgcccactggggacagaaggagaacac
ttcctgttcaccataggccatggcatggactcgggtcctcaatcttttga
gcacagtaatgggttctggatcttgggtaacaccactffitttgtttgtt
ttgcctcacaacaggaagataagtaacatcacttttttcctccatcctct
cacctaggtacccaagatattgtatttggagaagtagatcggtgagcagg
ggagcagcgtttgatcccccctgcctatcagcttcttctgtggagcctgt
tcctcactggaaattggcctctgtgtgtgtgtgtgtgtgtgtgtgtgtgt
gtatgttcatgtacacttggctgtcaggctttctgtgcatgtactaaaaa
aggagaaattataataaattagccgtcttgcggcccctaggcctaaa.

In some embodiments, the mutation is in any one of exons 1 (1-114 of SEQ ID NO:2), 2 (115-221 of SEQ ID NO:2), 3 (222-412 of SEQ ID NO:2), 4 (413-533 of SEQ ID NO:2), 5 (534-646 of SEQ ID NO:2), 6 (647-721 of SEQ ID NO:2), 7 (722-799 of SEQ ID NO:2), 8 (800-885 of SEQ ID NO:2), 9 (886-1005 of SEQ ID NO:2), 10 (1006-1135 of SEQ ID NO:2), 11 (1136-1231 of SEQ ID NO:2), 12 (1232-1315 of SEQ ID NO:2), or 13 (1316-1847 of SEQ ID NO:2) of the NDUFS2 gene.

In some embodiments, the mutation involves gene editing with CRISPR/Cas9 or Transcription activator-like effector nuclease (TALEN). Cas9 can be used to modify any desired genomic target provided that (1) the sequence is unique compared to the rest of the genome and (2) the sequence is located just upstream of a Protospacer Adjacent Motif (PAM sequence). The 3-5 nucleotide PAM sequence serves as a binding signal for Cas9 and this sequence is a strict requirement for Cas9-mediated DNA cleavage.

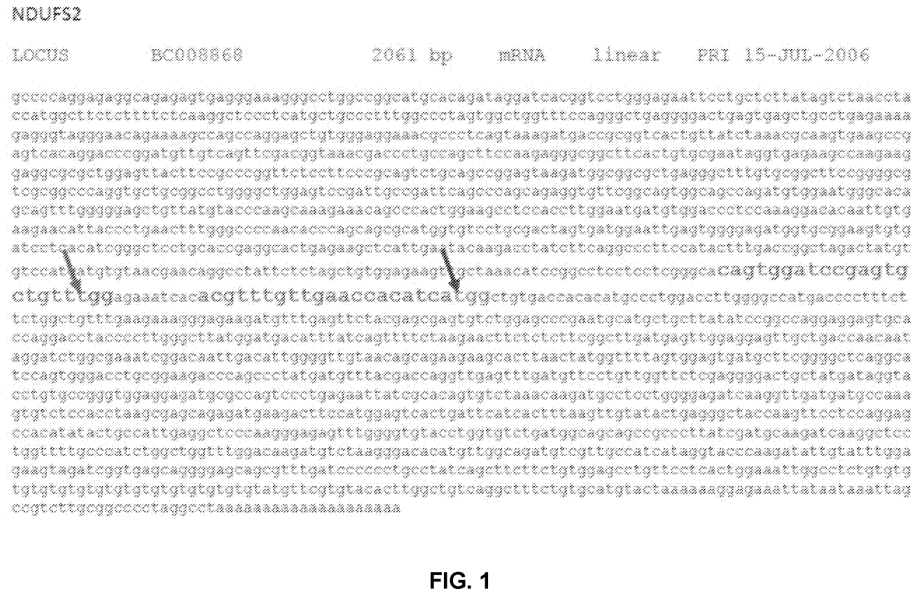

For example, the online idtdna program of Integrated DNA Technologies (Skokie, Ill.) predicted the 1002-bp site of SEQ ID NO:1 as the most reliable for mutating with 88% on-target specificity score. The relevant guide strand predicted by this program for this site is located right upstream of the PAM sequence of TGG (FIG. 1). Example guide strands to target this site include SEQ ID NOs:8 and 9. Alternatively, the online CRISPOR program predicted the 970-bp site of SEQ ID NO:1 as reliable for mutating with 87% on-target specificity score. Example guide strands to target this site include SEQ ID NOs:6 and 7.

While PAM sequences for the commonly used S. pyogenes Cas9 (3′-NGG) are abundant throughout the human genome, they are not always positioned correctly to target a particular gene. Furthermore, a target sequence may have high homology elsewhere in the genome. There are ways to circumvent this limitation, including: 1) the use of S. pyogenes Cas9 variants with varying PAM sequences, 2) use of Cas9 homologs derived from species other than S. pyogenes, and 3) use of non-Cas9 enzymes.

Cas9 is a nuclease that was first discovered as a component of the CRISPR system in Streptococcus pyogenes and has been adapted for utility in mammalian cells. RNA-guided Cas9 is able to efficiently introduce precise double-stranded breaks at endogenous genomic loci in mammalian cells with high efficiencies. Cas9 nucleases can be directed by short guide RNAs (sgRNA) to induce precise cleavage at endogenous genomic loci in human and mouse cells. Cas9 can also be converted into a nicking enzyme to facilitate homology-directed repair with minimal mutagenic activity. Lastly, multiple guide sequences can be encoded into a single CRISPR array to enable simultaneous editing of several sites within the mammalian genome, demonstrating easy programmability and wide applicability of the RNA-guided nuclease technology. Cas9 vectors express the Cas9 nuclease and the RNA sequences that guide the nuclease to its genomic target. Cas9 expression is driven by a choice of promoters and can be monitored by linked expression of green or red fluorescent proteins.

CRISPR/Cas9 mediated genome editing method disclosed herein comprises inserting a guide RNA into a NDUFS2 gene with CRISPR/Cas9 mediated genome editing to produce a plasmid. The cell lines are then subsequently produced using a host cell lines co-transfected with the plasmid produced by CRISPR/Cas9 mediated genome editing. The guide RNA used is capable of guiding CRISPR/Cas9 to the double strand break of host gene. Example guide RNAs include SEQ ID NO:6, 7, 8, or 9, or a functional variant thereof. The host cell lines can be any host cell that is capable of receiving recombinant vectors. Example host cell lines include HEK 293T, LNcaP, and PC-3.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Promoters controlling transcription from vectors in mammalian host cells may be obtained from various sources, for example, the genomes of viruses such as: polyoma, Simian Virus 40 (SV40), adenovirus, retroviruses, hepatitis-B virus and cytomegalovirus (CMV), or from heterologous mammalian promoters, e.g. beta actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. For instance, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region are active in all eukaryotic cell types, even if it is only expressed in a particular type of cell at a particular time. A promoter of this type for example is the CMV promoter (650 bases). Other example promoters are SV40 promoters, cytomegalovirus (full length promoter), and retroviral vector LTR.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. In some embodiments, the transcription unit also contain a polyadenylation region. One benefit of this region is that it increases the likelihood that the transcribed unit will be processed and transported like mRNA. The identification and use of polyadenylation signals in expression constructs is well established. For instance, homologous polyadenylation signals can be used in the transgene constructs. In certain transcription units, the polyadenylation region is derived from the SV40 early polyadenylation signal and consists of about 400 bases. In one embodiment, the polyadenylation signal is derived from the human growth hormone poly-A signal (hGH-pA). The transcribed units can contain other standard sequences alone or in combination with the above sequences improve expression from, or stability of, the construct.

The expression vectors can include nucleic acid sequence encoding a marker product. This marker product is used to determine if the gene has been delivered to the cell and once delivered is being expressed. For example, the marker genes can be the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of nutrients such as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

The host cells lines contemplated herein include eukaryotic cell lines suitable for producing a NDUFS2 mutant with disrupted Complex I activity. Contemplated cells include Hek293.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Abstract

Complex I is the largest and most intricate of the protein complexes of mitochondrial electron transport chain. This L-shape enzyme is constituted of two arms—a peripheral hydrophilic domain and a membrane-bound domain. The interfacial region between these two arms is known to be critical for binding of inhibitors and ubiquinone. NADH dehydrogenase [ubiquinone] iron-sulfur protein 2 (NDUFS2) is one of three protein subunits of the interfacial region. The NDUFS2 gene of human embryonic kidney cell line 293 was mutated by a CRISPR/Cas9 procedure. Compared to the parent, the resulting mutant grew slower (with 3.0 folds higher doubling time), displayed 4.0 folds lower complex I respiration, produced 15.8 folds less amount of ATP, and possessed 1.3 folds greater porous cell membrane. Treatment with idebenone, SBT-220, SBT-222, or elamipretide partially restored the ATP synthesis defects of the mutant. Overall, our results suggest that NDUFS2 is vital for growth, ATP synthesis and respiration of mammalian cells, and the respiratory defects of the NDUFS2 mutant can be corrected with use of potential complex I therapeutics.

### Introduction

Mitochondria are maternally inherited small organelle that play essential roles in energy production, reactive oxygen species (ROS) biology, apoptosis, and an array of intermediate metabolic pathways. Mitochondrion organelle is consisted of at least 1500 proteins. Genes encoding for 13 of these proteins are located in the mitochondrial genome, whereas, those encoding all other proteins are located in the nuclear genome. The functions of the nuclear genes are required for assembly/structure of oxidative phosphorylation (OXPHOS) enzyme complexes, biosynthesis of cofactors such as heme and iron-sulfur (Fe—S) clusters, and maintenance and expression of mitochondrial DNA.

The mitochondrion is compartmentalized by two lipid membranes; the inner membrane houses the OXPHOS enzymes, which are comprised of a series of five protein complexes. The collective organization of these protein complexes is described as an electron transport chain (ETC) since its function is to transfer electrons from electron donors to electron acceptors by way of reduction and oxidation (redox) reactions occurring simultaneously. This electron transfer mechanism facilitates the transfer of protons (H+ ions) across mitochondrial membrane creating an electrochemical proton gradient that drives the synthesis of adenosine triphosphate (ATP).

NADH:ubiquinone oxidoreductase (complex I; EC 1.6.5.3) is the first multimeric complex of the ETC and catalyzes the first step of the ETC in the mitochondrial OXPHOS system. Complex I is responsible for transferring electrons from nicotinamide adenine dinucleotide (NADH) to ubiquinone via flavin mononucleotide (FMN) and iron-sulfur clusters. The transfer of every two electrons from NADH to ubiquinone is coupled to the transfer of four protons across the mitochondrial inner-membrane. Structurally, the 1 MDa size complex I is the largest and most intricate of the enzyme complexes of the ETC. The mammalian complex I is assembled from at least 45 individual subunits of which seven subunits are mitochondrially-encoded while the rest of 38 subunits are nuclear-encoded [6].

Mammalian complex I is an L-shaped structure of which, its hydrophilic peripheral arm protrudes into the matrix of the organelle while its hydrophobic orthogonal arm is embedded in the lipid bilayer of the inner mitochondrial membrane. The interface region between the two arms is believed to be critical for the function of complex I enzyme as this (interface) region constitutes a large quinone or inhibitor binding pocket. The interface region is comprised of three major protein subunits, namely, NADH dehydrogenase [ubiquinone] iron-sulfur protein 2 (NDUFS2 or 49 kDa), NADH dehydrogenase [ubiquinone] iron-sulfur protein 7 (NDUFS7 or PSST), and NADH dehydrogenase 1 (ND1). Inhibitors in particular bind to the interfacial region between the peripheral and membrane arms. The large cleft formed by the NDUFS2 and NDUFS7 subunits is the site where ubiquinone is reduced by electrons from the terminal Fe—S cluster. Therefore, the interfacial juncture between the peripheral and membrane domains appears to be a “critical region” or a “hot spot” for the binding of inhibitors and ubiquinone. Nevertheless, the knowledge on organizational or functional aspects of the interfacial juncture of complex I is still scarce. In this study, we characterized the importance of NDUFS2 for in vitro growth, cell-membrane integrity, ATP synthesis, oxygen consumption, and complex I respiration of a human cell line.

Materials and Methods

Cell lines and culture conditions. Human embryonic kidney cell line 293 (HEK293) was provided. Media and reagents for growing and maintaining cells were purchased from Life Technologies Corporation (Carlsbad, Calif.). The cells were maintained in Dulbecco's Modified Eagle's medium (DMEM) supplemented with 10% (by volume) fetal bovine serum and 1% penicillin-streptomycin. Cells were sustained in a humidified incubator at 37° C. and 5% CO₂. A 0.25% trypsin-EDTA solution was used for detachment of cells.

One Shot™ Stbl3™ Chemically Competent cells of Escherichia coli strain (Life Technologies Corporation) was used for constructing the mutagenesis plasmid. Bacteria carrying the plasmids were maintained in Luria Bertani (LB; Sigma-Aldrich, St. Louis, Mo.) agar or broth, and sustained in a humidified incubator at 37° C. and 5% CO₂.

Design of CRISPR targeting components. The 2061 bp long nucleotide sequence of Homo sapiens NDUFS2 (GenBank locus ID BC008868) was used as the base for designing single guide RNA (sgRNA) sequences. Two separate online programs were used for choosing two separate sites for mutating. The online idtdna program of Integrated DNA Technologies (Skokie, Ill.) predicted the 1002-bp site of the forward strand as the most reliable for mutating with 88% on-target specificity score. The relevant guide strand predicted by this program for this site was ACGTTTGTTGAACCACATCA (SEQ ID NO:4) that is located right upstream of the protospacer adjacent motif (PAM) sequence of TGG (FIG. 1). Alternatively, the online CRISPOR program predicted the 970-bp site of the forward strand of NDUFS2 as reliable for mutating with 87% on-target specificity score. The relevant guide strand and PAM sequences predicted by this program for this site were CAGTGGATCCGAGTGCTGTT (SEQ ID NO:5) and TGG, respectively (FIG. 1). The top and bottom sgRNA sequences for these two sites (1002-bp site and 970-bp site) were designed and purchased from Integrated DNA Technologies (Table 1).

TABLE 1
Oligonucleotide sequences of guide
strands used in cloning

| Sequence name | Sequence (5′ to 3′) |
|---|---|
| 970-Fw-sgRNA-top | CACCgCAGTGGATCCGAGTGCTGTT |
| | (SEQ ID NO: 6) |
| 970-Fw-sgRNA-bottom | AAACAACAGCACTCGGATCCACTGc |
| | (SEQ ID NO: 7) |
| 1002-Fw-sgRNA-top | CACCgACGTTTGTTGAACCACATCA |
| | (SEQ ID NO: 8) |
| 1002-Fw-sgRNA-bottom | AAACTGATGTGGTTCAACAAACGTc |
| | (SEQ ID NO: 9) |

Construction of recombinant mutagenesis plasmid. The sgRNA oligonucleotides were resuspended at 100 μM concentrations. One μl volumes of each oligonucleotide was mixed with 8 μl of nuclease free water. Top and bottom oligonucleotides were annealed to each other on a thermocycler by using a slow anneal procedure that comprised of the following temperature conditions: 95° C. for 5 mins and 95° C. ramping down to 25° C. at 5° C. per min.

The annealed sgRNA strands were cloned into the plasmid px458 (Addgene, Watertown, Mass.). For this purpose, a digestion::ligation reaction was prepared by constituting 2.5 μl Fast Digest Buffer (Life Technologies Corporation), 16.5 μl nuclease-free water, 1.0 μl empty px458 plasmid at 1 μg/μl concentration (Addgene), 1.0 μl annealed oligos (prepared above), 2.5 μl 10×T4 DNA Ligase buffer (New England Biolabs, Ipswich, Mass.), 1.0 μl Fast Digest BpiI (Life Technologies), and 0.5 μl T4 DNA Ligase (New England Biolabs). The resulting 25 μl reaction was incubated at 37° C. for an hour followed by 70° C. for 30 min.

Competent E. coli Stbl3 cells were transformed with the digestion::ligation reaction, as described elsewhere (Ran F A, et al. Nat Protoc 2013, 8(11):2281-2308) and the protocol provided by the vendor. Briefly, 2 μl of digestion::ligation reaction was added into 20 μl of ice-cold chemically competent Stbl3™ cells, and the suspension was incubated on ice for 10 min, heat-shocked at 42° C. for 30 sec and returned to ice for 2 min. Subsequently, 100 μl of LB broth was added to the transformation reaction and the suspension was incubated at 37° C. for 1 h with shaking at 100 rpm. Aliquots of transformation reaction were plated on LB plates supplemented with ampicillin (Sigma-Aldrich) at 100 μg/ml. The cells on plates were incubated overnight at 37° C. On the following day, individual colonies were picked from plates and streaked on to fresh plates and incubated overnight. Plasmid DNA was extracted from these E. coli cells by using a QIAprep spin miniprep kit (Qiagen, Germantown, Md.) according to the manufacturer's instructions. The presence of the sgRNA segments within the constructed recombinant CRISPR plasmids was validated by DNA sequencing using the U6-Fwd primer (5′ GAGGGCCTATTTCCCATGATTCC 3′, SEQ ID NO:10). The plasmid carrying the sgRNA sequence of the 970-bp site of NDUFS2 was designated as px458-970 and the plasmid with the sgRNA sequence of 1002-bp site was named px458-1002.

Transfection of mammalian cells and isolation of recombinant mutant clones. HEK293 cells were transfected with the plasmids px458-970 and px458-1002 by using the Xfect Transfection Regent (Takara Bio USA Inc, Mountain View, Calif.), according to the manufacturer's instructions. Briefly, HEK293 cells grown to 65-75% confluence were harvested and plated onto 6-well plates at 0.5-1.0×10⁶ cells in 1 ml medium per well. One μg of plasmid DNA was suspended in 100 μl Xfect Reaction Buffer and 1.5 μl of Xfect polymer was added to the buffer::DNA suspension. The reaction was incubated at room temperature for 10 min and added into the medium carrying HEK293 cells in 6-well plates. After 24 to 48 h incubation, cells in plates were harvested by treatment with trypsin-EDTA, suspended in fresh medium at a ratio of 0.5 cells:100 μl medium (approximately 60 cells in 12 ml medium), and plated in 96-well plates at 100 μl/well. After 5-7 days of incubation, the colonies were inspected for clonal appearance: the wells with rounded colonies radiating from a central point indicated the ones seeded with a single cell. The cells were returned to the incubator and allowed to expand for 2 to 3 additional weeks. A total of 43 wells were seen to carry colonies radiating from a central point, and therefore were chosen for further work. Cells were harvested from these 43 individual wells (clones). Portions of harvested cells were saved in 10% dimethyl sulfoxide (DMSO; Sigma-Aldrich) in a liquid N dover, a portion was used for extraction of cellular protein, while another portion used for extracting genomic DNA.

Mutant validation by western immuno-blotting. Protein was extracted from harvested cells by using the M-PER Mammalian Protein Extraction Reagent (ThermoFisher Scientific, Rockford, Ill.), and protein yields were quantified by using the Pierce BCA Protein Assay Kit (Life Technologies Corporation, Grand Island, N.Y.), according to the protocol provided by the manufacturer. Western Immuno-blotting was performed by using the standard procedures. Briefly, protein preps suspended in Laemmli Sample Buffer (BioRad Laboratories Inc, Hercules, Calif.) were boiled for 5 min, spun down at 10,000×g for 10 min, and run in 4-20% Mini-PROTEAN protein gels (BioRad). Proteins from gels were transferred to IPFL10100|Immobilon-FL PVDF Membrane (EMD Millipore, Burlington, Mass.), and the membranes were blocked with Odyssey® Blocking Buffer (LI-COR Biosciences, Lincoln, Nebr.), incubated at 4° C. overnight with rabbit polyclonal antibodies to NDUFS2 (1:1000 in blocking buffer) (Cat #PA522364; Life Technologies Corporation, Carlsbad, Calif.) or rabbit polyclonal antibodies to β-Actin (1:4000) (Cat #ab8227, Abcam Inc, Cambridge, Mass.), washed with phosphate buffered saline (PBS; Life Technologies Corporation), and incubated for 1 h with the secondary antibody IRDye® 800CW Donkey anti-Rabbit IgG (H+L) (Cat #P/N 925-32213; LI-COR Biosciences). The protein bands were visualized by using the ODYSSEY CLx imaging system (LI-COR Biosciences).

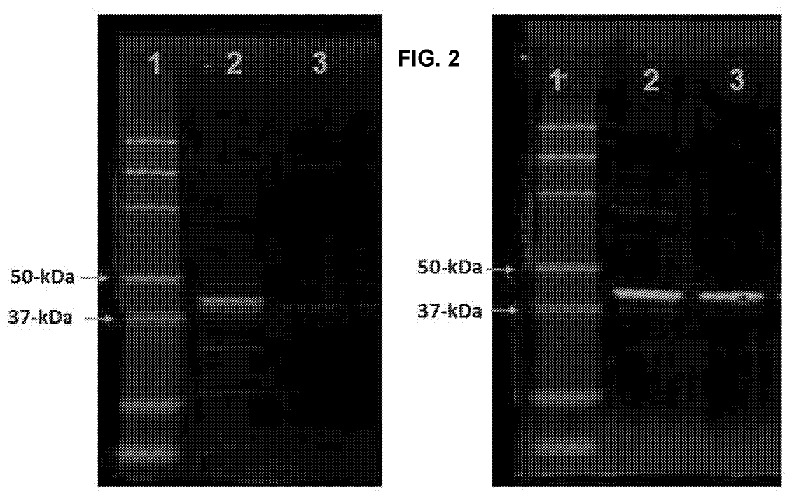

Six out of 43 clones examined by western immuno-blotting were found missing a protein band of approximately 49 kDa when the respective blots were incubated with the primary antibody to NDUFS2 (FIG. 2). This observation suggests that these six clones are missing a protein equivalent in size to that of NDUFS2. Nevertheless, all the tested clones produced an approximately 42 kDa size band when the respective blots were incubated with the primary antibody to β-Actin (the house-keeping protein) (FIG. 2). One of the six clones missing NDUFS2 was chosen for further work and designated as HEK293ΔNDUFS2.

In vitro growth of strains in culture medium. Parent strain HEK293 and the mutant HEK293ΔNDUFS2 grown to 75% confluence in 75 cm² flasks were harvested and resuspended in growth media. Aliquots of 50,000 cells were introduced into 25 cm² flasks each carrying 5 ml growth media. Cultures were incubated for 4 or 6 days, cells harvested by trypsin-EDTA treatment, and cell numbers quantified in triplicates. Doubling times of the cell lines were calculated.

Mitochondrial respiration measurements with Oroboros O2K respirometry. Oxygen consumption rate (OCR) of strains was determined using protocols described elsewhere (Alleman R J, et al. Am J Physiol Heart Circ Physiol 2016, 310(10):H1360-1370; Goswami I, et al. Biophys J 2018, 114(12):2951-2964). Briefly, the parent type HEK293 and the mutant HEK293ΔNDUFS2 grown for eight days in 225 cm² flasks for 75-80% confluence were harvested by treatment with trypsin-EDTA, centrifuged at 300×g for 3 min, and resuspended in assay buffer Z (105 mM 2-(N-Morpholino)ethanesulfonic acid potassium salt, 30 mM KCl, 10 mM KH₂PO₄, 5 mM MgCl₂-6H₂O, 0.5 mg/mL bovine serum albumin, 20 mM creatine, 1 mM EGTA [pH 7.1] in dH₂O). Approximately one million cells in 2 ml volumes were then introduced into the Oroboros O2K respiration chamber, which is equipped with calibrated oxygen measuring polarographic sensors. The oxygen signals were allowed to stabilize before closing the chamber to the outside air while chamber temperature was maintained at 37° C. Subsequently, the OCR was recorded after injection with none (basal), 2 mM of malate plus 10 mM glutamate (G/M), 5 mM of adenosine diphosphate (ADP), and 1.0 μM protonophore Carbonyl cyanide 4-(trifluoromethoxy)phenylhydrazone (FCCP).

ATP synthesis and cell-membrane integrity of strains. Potential mitochondrial dysfunction of strains was assessed by using the Mitochondrial ToxGlo Assay (Promega Corporation, Madison, Wis.), according to the procedure described by the manufacturer. This assay is based on the measurements of biomarkers associated with changes in cell membrane integrity and cellular ATP levels. Briefly, the parent and mutant cells grown in flasks were harvested by trypsinization and resuspended in fresh medium. Approximately 10,000 cells/well were added to a 96-well plate and incubated overnight. A working stock of antimycin A, which is a mitochondrial toxin (complex III inhibitor) was prepared at a concentration of 150 μM in a glucose-free DMEM medium (Sigma-Aldrich) supplemented with 10 mM galactose (Sigma-Aldrich). Serial dilutions of the mitochondrial toxin were prepared in a separate 96-well plate (in galactose containing medium). Overnight grown cells were washed with glucose-free galactose containing medium, and fresh (galactose containing medium) was added into wells at 50 μl/well. Fifty μl volumes of the serial dilutions of toxin were transferred from the stock plate to the wells in cell culture plate. The wells carrying the vehicle controls received the medium with no toxin. The plate was incubated at 37° C. for 90 min. Twenty μl volumes of 5× Cytotoxicity Reagent were added to each well, the plate was incubated at 37° C. for further 30 min, and the fluorescence was measured at 475 nm_{EX}/500-550 nm_{EM}, by using a Glomax Discover plate reader (Promega Corporation). The assay plate was equilibrated to room temperature, 100 μl ATP Detection Reagent was added to each well, and luminescence was measured by using the same plate reader.

Analysis of mitochondrial distribution by fluorescence microscopy. Distribution of mitochondrial organelle within the cytoplasm were examined by MitoTracker staining, as described elsewhere (Dell'Anna M L, et al. Sci Rep 2017, 7(1):13663). Briefly, approximately 10,000 cells were plated on MatTek glass cover slips (MatTek Corporation, Ashland, Mass.), incubated 48 h at 37° C. and 5% CO₂ and strained with MitoTracker Green FM (Life Technologies Corporation, Grand Island, N.Y.), according to the manufacturer's instructions. Fluorescence signals were analyzed by recording stained 63× images using a confocal laser scanning microscope.

Restoring (rescuing) the altered/lost phenotypes of the mutant with the use of potential complex I therapeutics. Effectiveness of the potential complex I therapeutics in restoring the metabolic defects of the mutant HEK293ΔNDUFS2 was evaluated. For this purpose, the mutant cells were treated with the therapeutic idebenone or its analog SBT-220 and change in ATP synthesis was examined. In this procedure, the mutant cells were suspended in culture media supplemented with 0, 1, or 10 μM of idebenone, conjugate SBT-220, conjugate SBT-222, or elamipretide, and plated in 96 well plates at 2,000 cells/well. Cells suspended in DMSO served as controls. Fresh media (with or without therapeutics) were added on days 0, 3 and 5. Cellular ATP synthesis was assessed on day 6 of incubation by using the Mitochondrial ToxGlo Assay, as described above. The responses of ATP synthesis in the treatment groups were normalized to that of the control group.

Statistical analyses. The average and standard error of in vitro growth of strains, membrane porousness and ATP synthesis were calculated by using Microsof Excel 2016 program (Microsoft, Redmond, Wash.) and graphed using GraphPad Prism 7 (GraphPad Software, San Diego, Calif.). The data from Oroboros were tabulated and graphed by using GraphPad Prism 7. Student's t-tests were performed by using GraphPad Prism 7 to compare the means of the parent control versus the mutant in terms of Oroboros OCR, ATP synthesis, membrane porousness, and growth in media. Student's t-tests were also used to compare the means of the DMSO control versus the treatment with idebenone, SBT-220, SBT-222, or elamipretide in terms of ATP synthesis in the mutant cell line. The mean differences between the parent versus mutant or DMSO-control versus drug-treatment were considered statistically significant at p<0.05.

Results

Construction of recombinant mutant HEK293.6NDUFS2. Two separate sites of the gene NDUFS2 on HEK293 genome were targeted for mutating based on the predictions made by two separate online CRISPR guide tools. One of the tools, the online program of Integrated DNA Technologies, predicted the 1002-bp site of the forward strand of the gene as the most reliable for mutagenesis with an on-target specificity score of 88% (FIG. 1). The recombinant plasmid constructed for mutating this site was named px458-1002. A second site for mutagenesis was chosen based on the prediction of the other online guide tool named CRISPOR. This program predicted the 970-bp site of the forward strand of the gene as reliable for mutagenesis with 87% on-target specificity score (FIG. 1). The plasmid constructed for mutating this second site was designated px458-970.

From the transfection of strain HEK293 with the mutagenesis plasmid px458-1002, 22 individual clones were seen growing from single individual cells in wells of the 96 well plates. Out of these 22 clones, six were found missing the approximately 49 kDa protein band (FIG. 2) suggesting that the gene NDUFS2 was disrupted in these clones. The remaining 16 clones produced protein profiles exactly similar to those of the parent strain HEK293 suggesting that mutagenesis did not occur in those clones. One of the clones missing the 49 kDa protein band was chosen for further assays and designated HEK293ΔNDUFS2. From the transfection of HEK293 cells with the mutagenesis plasmid px458-970, 21 individual clones were seen growing from single cells. According to western immuno-blotting results, all 21 of these clones produced protein profiles exactly similar to the parent strain HEK293 suggesting that none of these clones carried an expected mutation in NDUFS2 gene.

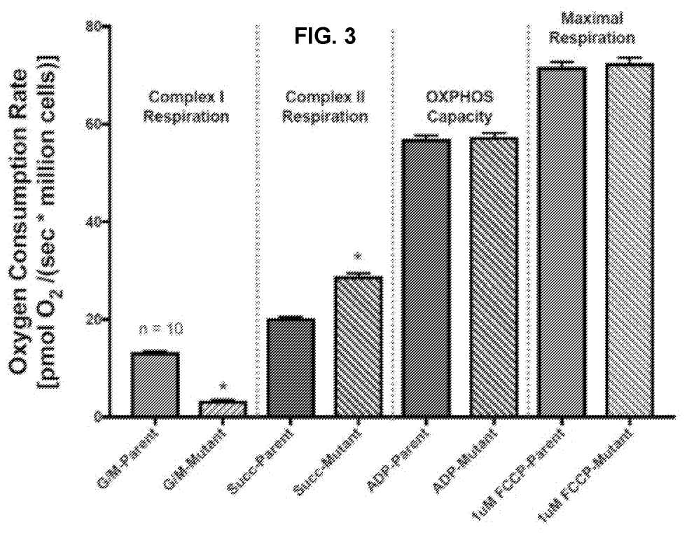

Mitochondrial respiration of strains. Respiration was measured by using Oroboros Oxygraph 2k (FIG. 3). Oxygen consumption was collected at each step along electron transport system. Compared to that in parent HEK293, in mutant HEK293ΔNDUFS2, complex I respiration reduced significantly with a 4.0-fold lower oxygen consumption rate (p<0.0001). This observation highlights the vitality of the NDUFS2 functions for complex I respiration. Nevertheless, complex II respiration increased in the mutant with a 1.4-fold greater oxygen consumption rate than in the parent (p=0.001). This finding suggests a possible compensatory regulation by the complex II in a scenario of a functional deficiency of complex I (FIG. 3).

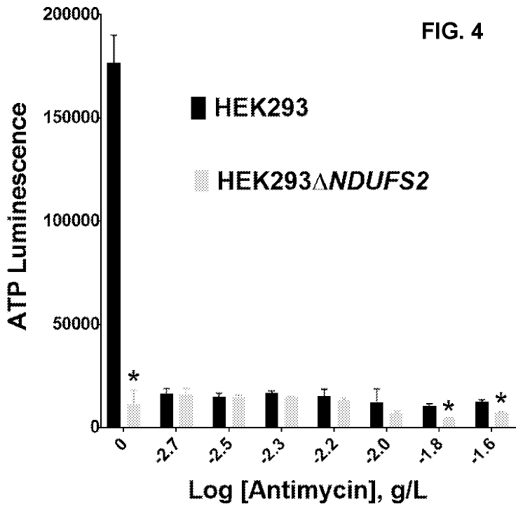

ATP synthesis. Total ATP synthesis of cells was measured by using the Mitochondrial ToxGlo Assay (FIG. 4). The ATP detection reagent of the assays consisted of ATPase inhibitors, a luciferin-containing formulation for ATP detection, and a thermo-stable luciferase. Addition of the ATP detection reagent into cells caused cell lysis and generation of a luminescent signal proportionate to the amount of ATP present. Luminescence data indicated that the parent strain produced 15.8 times more ATP than the mutant did (p=0.0005) at the absence of respiratory inhibitors. When the complex Ill respiratory inhibitor Antimycin A was added to the reaction media, ATP synthesis ability of both the parent as well as the mutant declined drastically, but the decline was up to 2.2 folds greater in the mutant compared to the parent (p=0.0014).

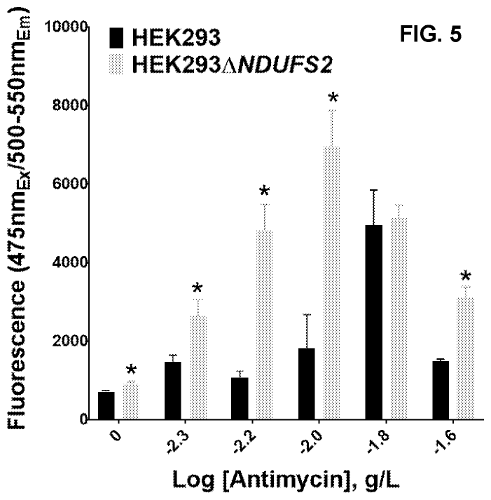

Cell-membrane integrity. Potential mitochondrial dysfunction of strains was also assessed by using the Mitochondrial ToxGlo Assay, which is based on the measurements of biomarkers associated with changes in cell membrane integrity (FIG. 5). Porousness or integrity of the cell membrane was examined by detecting the presence or absence of a distinct protease activity associated with necrosis using a fluorogenic peptide substrate. The fluorogenic substrate is generally unable to cross the intact membrane of live cells and therefore gives insignificant signal with viable cells relative to non-viable cells. Thus, the cell membrane integrity measured a “dead cell protease activity”. Fluorescence data suggested that the membrane porousness (membrane weakness) of the mutant was 1.3 folds higher than that of the parent (p=0.0221) at the absence of any respiratory inhibitors. Addition of Antimycin A to the reaction medium made the membranes of both the parent as well as the mutant more porous. Nevertheless, the cell membrane of the mutant became up to 4.5 folds more porous (weaker) than that of the parent as a result of antimycin A treatment (p=0.0013).

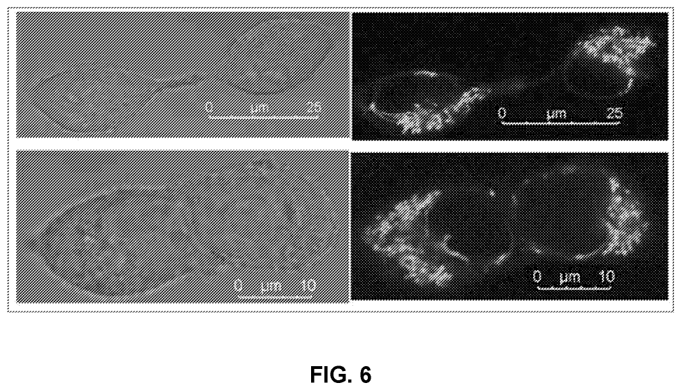

Distribution of mitochondria in cell cytoplasm. Confocal micrographs displayed that both the parent (FIG. 6 top panel) as well as the mutant (FIG. 6 bottom panel) possessed a well-organized network of mitochondria evenly dispersed throughout the cell cytoplasm and away from the nucleus. Our observations were not sufficient enough to suggest any impact of the disruption of NDUFS2 on mitochondrial network assembly.

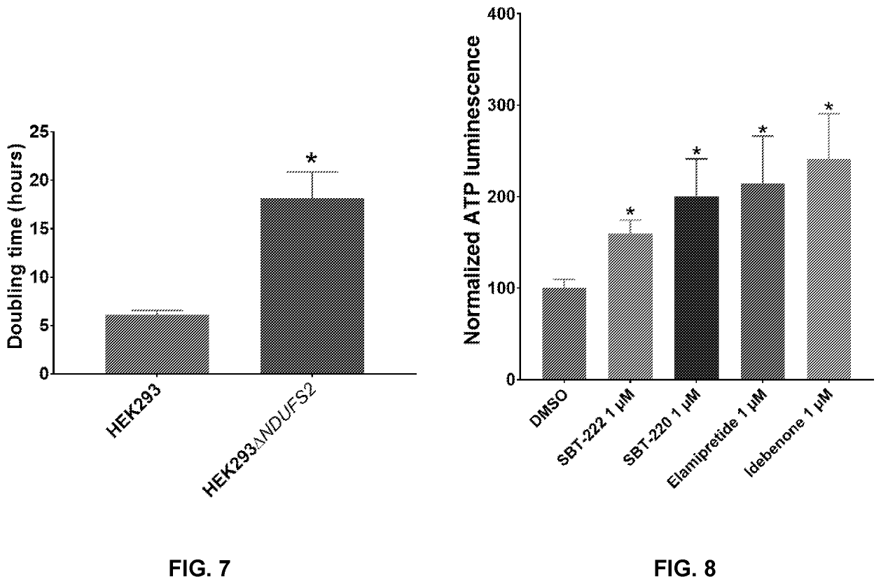

In vitro growth of strains in culture medium. Total number of cells in flasks carrying the parent or the mutant was calculated on the 4th or 6th days of incubation in media. After four days of incubation, the number of cells recovered from the mutant culture was 38% of that from the parent strain culture. After six days of incubation, the number of cells recovered from the mutant was 18% of that from the parent. These observations suggest that in culture media, the mutant grow 62% (p=0.019) and 82% (p=0.027) slower than the parent strain at 4 and 6 days of incubation, respectively. The doubling time of the mutant between the days 0 and 4 was 3.0 folds bigger than the doubling time of the parent during the same duration of growth (p=0.0477) (FIG. 7).

Restoring (rescuing) the altered/lost phenotypes of the mutant by using the potential complex I therapeutics. Compared to the mutant cells treated with DMSO, the mutant cells treated with 1 μM concentrations of idebenone, SBT-220, SBT-222, or elamipretide respectively produced 2.4 folds (p=0.0101), 2.0 folds (p=0.0266), 1.6 folds (p=0.0014), or 2.1 folds (p<0.0056) greater amounts of ATP (FIG. 8). Treatment with 10 μM concentrations of these drugs failed to improve the ATP synthesis defects of the mutant.

### Discussion

Since the primary function of mitochondria is to convert the energy stored in food molecules into ATP, mitochondrial diseases involve organs or tissues that have greater energy demands, such as, brain, retina, heart, muscles, liver, and endocrine systems. When mitochondria are unable to produce amounts of energy sufficient for normal tissue function, a threshold is crossed, and cell degeneration is started leading to disease symptoms (Wallace D C, et al. Genes Dev 2009, 23(15):1714-1736). Symptoms resulting from mitochondrial dysfunction include poor growth, diseases in heart, liver, kidney and nervous system, disorders in gastrointestinal and respiratory systems, problems in vision and hearing, lack of muscle coordination, muscle weakness, autonomic dysfunction, and dementia (Blau N, M. et al. Springer 2014, ISBN 978-3-642-40337-8:339). Acquired conditions resulting from mitochondrial dysfunction include, cardiovascular disease, diabetics, cancer, aging, Huntington's disease, Alzheimer's disease, Parkinson's disease, anxiety disorders, bipolar disorder, schizophrenia, sarcopenia, and chronic fatigue syndrome (Stork C, et al. Mol Psychiatry 2005, 10(10):900-919; Pieczenik S R, et al. Exp Mol Pathol 2007, 83(1):84-92; Nierenberg A A, et al. Aust N Z J Psychiatry 2013, 47(1):26-42).

Mutations in mitochondrial as well as nuclear genes lead to mitochondrial diseases. Nevertheless, only 15-20% of patients suffering from mitochondrial diseases carry mutations in mitochondrial genome. Of the remainder of mitochondrial patients, mutations are harbored in nuclear genome (Kirby D M, et al. Twin Res Hum Genet 2008, 11(4):395-411). Isolated complex I deficiency is the most frequently observed OXPHOS defect among children with mitochondrial disease (Kirby D M, et al. Neurology 1999, 52(6):1255-1264; Triepels R H, et al. Am J Med Genet 2001, 106(1):37-45). Clinical presentations associated with isolated complex I include hepatopathy, cardiomyopathy, fatal congenital lactic acidosis, and Leigh syndrome. The common neurological features of Leigh syndrome include central hypopnea, dystonia, hypotonia, dysphagia, and myopathy (Distelmaier F, et al. Brain 2009, 132(Pt 4):833-842). Mutations in mitochondrial genes are thought to account for approximately 25% of the complex I cases (Bugiani M, et al. Biochim Biophys Acta 2004, 1659(2-3):136-147; Lebon S, et al. J Med Genet 2003, 40(12):896-899) suggesting that nuclear gene defects must be responsible for the rest of the observed cases.

Heterozygous NDUFS2 nuclear complex I mutations were found responsible for isolated complex I deficiency in four Caucasian patients (Tuppen H A, et al. Brain 2010, 133(10):2952-2963). NDUFS2 mutations were also found in patients with cardiomyopathy and encephalomyopathy (Loeffen J, et al. Ann Neurol 2001, 49(2):195-201). The nuclear gene NDUFS2 encodes for NDUFS2 protein, which is a subunit of the OXPHOS enzyme complex I that is harbored on the inner mitochondrial membrane. This subunit together with NDUFS7 and ND1 form the critical complex I interface region that constitutes a large quinone or inhibitor binding pocket (Efremov R G, et al. Nature 2010, 465(7297):441-445; Hunte C, et al. Science 2010, 329(5990):448-451). Moreover, a cleft formed by NDUFS2 and NDUFS7 serves as the site where ubiquinone is reduced by electrons from the terminal Fe—S cluster. Thus, NDUFS2 is anticipated to perform a major role in assembly of the complex I on mitochondrial inner membrane and also in generating the proton gradient necessary to produce ATP.

Majority of studies aimed at characterizing the structure and organization of the complex I, its interfacial juncture, as well as the importance of this juncture in ubiquinone/inhibitor binding processes, has been done using microbial models, including Thermus thermophiles (Efremov R G, et al. Nature 2010, 465(7297):441-445; Sazanov L A, et al. Science 2006, 311(5766):1430-1436) and Yarrowia lipolytica (Hunte C, et al. Science 2010, 329(5990):448-451). Nevertheless, the knowledge on organizational or functional aspects of the interfacial juncture of complex I in mammalian systems is still scarce. In this backdrop, we investigated the functions of NDUFS2 of mammalian cell line HEK293.

Two separate sites of HEK293 genome were targeted for mutating. The efforts to mutate the 1002-bp site of NDUFS2 gene successfully produced six clones with disrupted NDUFS2 expression. The online guide tool of Integrated DNA Technologies had predicted this site as reliable and specific for mutating this gene. Nevertheless, the efforts to mutate the 970-bp site of the gene failed to produce any clones with disrupted NDUFS2 expression. The online guide tool named CRISPOR had predicted this (second) site as suitable for mutagenesis. The failure to mutate the 970-bp site can be attributed to two possible reasons: (i) the sequence in this site may not be compatible for the CRISPR/Cas9 cleaving insertion/deletion (indel) mechanism; or (ii) the disruption in this site of the gene may be lethal for the cell line, and therefore, the clones may not have survived upon mutagenesis.

Disruption of synthesis of the 49-kDa size NDUFS2 protein in the mutant was confirmed by western immune-blotting procedure. Nevertheless, our efforts to confirm the mutation in the NDUFS2 gene or to locate the specific site of the disruption of this gene were not successful since it was unable to PCR amplify the targeted DNA region. Multiple primer combinations, buffers, MgCl₂ concentrations, template DNA amounts, and annealing temperatures were used unsuccessfully in PCR reactions. Moreover, sequencing of the obtained PCR products failed to produce valid results.

Mutation in NDUFS2 disrupted the complex I respiration as evidenced by Oroboros Oxygraph 2k observations. This was anticipated since NDUFS2 is a subunit of complex I and a constituent of the interfacial region of this complex. Nevertheless, the mutant exhibited a greater complex II respiration than the parent strain possibly because the complex II attempts to function at a greater rate in order to compensate the overall oxygen consumption. Mitochondrial ToxGlo™ assay results revealed that the mutant produced significantly less amount of ATP than the parent. Overall, the findings of the O2K and ToxGlo™ assays strongly indicated that the OXPHOS respiration of the cell line got severely impaired due to the mutation of NDUFS2 gene. These findings also suggest that the functions of NDUFS2 enzyme subunit is vital for the overall energy production functions of the ETC.

Peralta et al., (Peralta S, et al. Hum Mol Genet 2014, 23(6):1399-1412) constructed a knockout mouse strain by inactivating the nuclear gene Ndufa5 that encodes for the NDUFA5 protein of complex I. The levels of NDUFA5 in the resulting strain was reduced to 25% of the parent strain. Moreover, the complex I activity of homogenates of the knockout strain was reduced to 60% of the parent. Similar to that in the Ndufa5 knockout mice, the complex I respiration in our NDUFS2 cell line was also reduced. Nevertheless, reduction in complex I respiration in our NDUFS2 mutant cell line was much greater (4 folds). This difference in complex I respiration between the knockout mouse strain and our cell line can be attributed to a number of reasons. In the Ndufa5 knockout mice, the expression of NDUFA5 was reduced to 25% whereas in our cell line the expression of NDUFS2 was reduced to an undetectable level. Moreover, the NDUFA5 is a protein located in the peripheral arm of the complex I whereas NDUFS2 is located in the interfacial region between peripheral and membrane arms of the same complex. As described above, the interfacial region plays a greater role in binding of ubiquinone or inhibitors. Thus, disruption of a protein in the highly vital interfacial region must have caused to a greater damage to the enzyme activity of complex I. It is possible that the role played by NDUFS2 is greater than the role played by NDUFA5 in complex I activity.

Ingraham et al., (Ingraham C A, et al. Mitochondrion 2009, 9(3):204-210) created a mouse model harboring a point mutation in Ndufs4 gene. In homozygous mutant fetuses (NDUFS4^{-/-}), embryonic lethal phenotype was observed. In heterozygous animals, decrease in complex I activity of around of around 25-30% was observed. Quintana et al., (Quintana A, et al. Proc Natl Acad Sci USA 2010, 107(24):10996-11001) inactivated the Ndufs4 selectively in neurons and glia of mice. Complex I dependent oxygen consumption in brain tissue of these mice reduced by approximately 50% compared to the control mice. Ke et al., (Ke B X, et al. Proc Natl Acad Sci USA 2012, 109(16):6165-6170) generated a complex I deficient mouse model by knocking down Ndufs6 gene expression using a gene-trap embryonic cell line. Complex I activity in heart tissues of these mice reduced to approximately 10% of the control mice.

The ToxGlo™ assay results also indicated that the cell membrane of the mutant was more porous than that of the parent. This finding attributes a relationship between the mitochondrial functions and cell-membrane integrity. Treatment with antimycin A made the membranes of both the parent as well as the mutant even more porous (weaker). Nevertheless, the effect of antimycin A on membrane integrity of the mutant was greater than that of the parent. These observations suggest that NDUFS2 plays an important role in maintenance of the cell membrane integrity. Confocal images showed that of both the parent as well as the mutant, mitochondrion organelles localized evenly dispersed within the cytoplasm around the nucleus. Great amounts of these organelles were found densely associated in the cytoplasm away from the nucleus. Our observations failed to suggest any impact of the disruption of NDUFS2 on the assembly of mitochondrial network.

The mutant HEK293ΔNDUFS2 grew significantly slower than the parent HEK293 in culture media. It is possible that in the mutant, poor oxygen consumption, reduced ATP synthesis, and weaker membrane integrity, all contributed to impair the metabolic activities, and hence to disrupt the cell proliferation.

Abnormal mitochondrial function has been implicated to consequent impairment of OXPHOS energy production, which eventually leads to heart failure. Thus, there is a growing interest in mitochondrial function as a therapeutic target (Brown D A, et al. Nat Rev Cardiol 2017, 14(4):238-250; Bayeva M, et al. J Am Coll Cardiol 2013, 61(6):599-610; Ingwall J S, et al. Circ Res 2004, 95(2):135-145). Quinones are organic compounds found in nature and function as coenzymes, antioxidants, signaling molecules and vitamins (Haefeli R H, et al. PLoS One 2011, 6(3):e17963; Erb M, et al. PLoS One 2012, 7(4):e36153; Heitz F D, et al. PLoS One 2012, 7(9):e45182). Coenzyme Q₁₀ also known as CoQ₁₀ or ubiquinone is one of the physiological quinones that is ubiquitous in animals and bacteria (Haefeli R H, et al. PLoS One 2011, 6(3):e17963). For cellular energy production, it is essential to create an electrochemical proton gradient between the inner-membrane and the matrix of mitochondria. This membrane potential is generated or maintained by shuttling electrons from complexes I and II to complexes III and IV of the mitochondrial ETC. The membrane potential generated by this electron transfer/shuttling process eventually support the production of ATP (Beal M F, et al. Biofactors 2003, 18(1-4):153-161; McCarthy S, et al. Toxicol Appl Pharmacol 2004, 201(1):21-31; Gueven N, et al. Mitochondrion 2017, 36:7-14). CoQ10 is responsible for shuttling electrons between complexes I, II and III. This quinone contains a large hydrocarbon side chain that comprises ten isoprenyl units. The highly lipophilic nature of this isoprenyl tail leads to an extremely reduced solubility of CoQ10 in aqueous environments resulting in poor absorption at cellular sites (McCarthy S, et al. Toxicol Appl Pharmacol 2004, 201(1):21-31; Gueven N, et al. Redox Biol 2015, 4:289-295; Fash D M, et al. Bioorg Med Chem 2013, 21(8):2346-2354). Thus, the highly lipophilic isoprenyl tail limits the effectiveness of CoQ10 as a potential therapeutic (Haefeli R H, et al. PLoS One 2011, 6(3):e17963). Idebenone is a short-chain benzoquinone with greater hydrophilicity (Erb M, et al. PLoS One 2012, 7(4):e36153). Therefore, the use of this compound as a therapeutic for conditions associated with oxidative stress and mitochondrial dysfunction is investigated (Haefeli R H, et al. PLoS One 2011, 6(3):e17963; Erb M, et al. PLoS One 2012, 7(4):e36153; Heitz F D, et al. PLoS One 2012, 7(9):e45182). In clinical trials, idebenone has shown promise against many neuromuscular and mitochondrial disorders, including Leber's hereditary optic neuropathy (LHON) (Haefeli R H, et al. PLoS One 2011, 6(3):e17963; Erb M, et al. PLoS One 2012, 7(4):e36153; Heitz F D, et al. PLoS One 2012, 7(9):e45182), Friedreich's ataxia (FRDA) (Palecek T, et al. Curr Pharm Des 2015, 21(4):491-506; Strawser C J, et al. Expert Rev Neurother 2014, 14(8):949-957; Lynch D R, et al. Arch Neurol 2010, 67(8):941-947), Duchenne muscular dystrophy (DMD) (Buyse G M, et al. Neuromuscul Disord 2011, 21(6):396-405; Buyse G M, et al. Pediatr Pulmonol 2017, 52(4):508-515; McDonald C M, et al. Neuromuscul Disord 2016, 26(8):473-480; Buyse G M, et al. Lancet 2015, 385(9979):1748-1757), and multiple sclerosis (MS) (Fiebiger S M, et al. J Neuroimmunol 2013, 262(1-2):66-71). The effectiveness of idebenone was evaluated in correcting the respiratory deficiency of the NDUFS2 mutant. Treatment with idebenone or its conjugates SBT-220 and SBT-222 at 1 μM concentration restored the ATP synthesis of the mutant to some extent. These observations suggest that the effects of NDUFS2 disruptions can be rescued at least to some degree by using the potential complex I therapeutics. Thus, the constructed mutant HEK293ΔNDUFS2 can be useful as a platform to evaluate the effectiveness of the novel therapeutics for treatment of complex I deficiencies.

Elamipretide, also known as bendavia, is an aromatic-cationic tetra-peptide that has entered clinical trials (Ajith T A, et al. World J Cardiol 2014, 6(10):1091-1099; Szeto H H. Br J Pharmacol 2014, 171(8):2029-2050; Daubert M A, et al. Circ Heart Fail 2017, 10(12)). This cell-permeable peptide is targeted to mitochondria via cardiolipin. In animal models, elamipretide has shown to improve energetics while decreasing ROS production, most likely by stabilizing the mitochondrial membrane and cytochrome c (Zhao K, et al. J Biol Chem 2004, 279(33):34682-34690; Birk A V, et al. Br J Pharmacol 2014, 171(8):2017-2028). Elamipretide is capable of entering the tissues within minutes after treatment and produced cardioprotective effects in ischemia/reperfusion injury in animal models (Dai W, et al. J Cardiovasc Pharmacol 2014, 64(6):543-553; Sabbah H N, et al. Circ Heart Fail 2016, 9(2):e002206; Shi J, et al. Life Sci 2015, 141:170-178; Cho J, et al. Coron Artery Dis 2007, 18(3):215-220). This tetra-peptide also improved mitochondrial oxygen consumption, activities of complexes I and IV, and activities of complex IV in failing human hearts (Chatfield K C, et al. JACC Basic Transl Sci 2019, 4(2):147-157). In this study, treatment with elamipretide partially restored the ATP synthesis of the mutant.

### Conclusions

The functions of NDUFS2 are vital for growth, complex I respiration, ATP synthesis, cell-membrane integrity, and in vitro growth of HEK293. The respiratory effects of NDUFS2 disruption can be partially restored by treatment with potential complex I therapeutics idebenone, idebenone conjugates, or elamipretide. The constructed mutant may be useful as a platform to study the usefulness of novel complex I therapeutics.

### Example 2

Materials and Methods

Cell lines and culture conditions. Human embryonic kidney cell line 293 (HEK293) was provided. Media and reagents for growing and maintaining cells were purchased from Life Technologies Corporation (Carlsbad, Calif.). The cells were maintained in Dulbecco's Modified Eagle's medium (DMEM) supplemented with 10% (by volume) fetal bovine serum and 1% penicillin-streptomycin. Cells were sustained in a humidified incubator at 37° C. and 5% CO₂. A 0.25% trypsin-EDTA solution was used for detachment of cells. One Shot™ Stbl3™ Chemically Competent cells of Escherichia coli (Life Technologies Corporation) were used for constructing the mutagenesis plasmid. Bacteria carrying the plasmids were maintained in Luria Bertani (LB; Sigma-Aldrich, St. Louis, Mo.) agar or broth, and sustained in a humidified incubator at 37° C.

Construction and validation of NDUFS2 mutant. Single guide RNA (sgRNA) sequences were designed, purchased from Integrated DNA Technologies, and cloned into plasmid px458 (Addgene, Watertown, Mass.). HEK293 cells were transfected with recombinant px458 plasmid using transfection reagent Xfect (Takara Bio USA Inc, Mountain View, Calif.). Clones with mutation in NDUFS2 were picked using procedures described elsewhere (Ran F A, et al. Nat Protoc 2013, 8(11):2281-2308) and validated by Western Immuno-blotting using rabbit polyclonal antibody to NDUFS2 (Cat #PA522364; Life Technologies Corporation, Carlsbad, Calif.) and rabbit polyclonal antibodies to β-Actin (Cat #ab8227, Abcam Inc, Cambridge, Mass.). A clone missing SDHD was chosen for further work and designated as HEK293ΔNDUFS2 (FIG. 1).

Cell growth and metabolism. To measure the cell proliferation of parent HEK293 and mutant HEK293ΔNDUFS2, aliquots of 50,000 cells were introduced into 75 cm² flasks each carrying 25 ml growth media and cell numbers were quantified in triplicates after 4 and 6 days of incubation. Extracellular Acidification Rate (ECAR) was determined with an Agilent Seahorse XFe96 analyzer (Ryall J G. Methods Mol Biol 2017, 1556:245-253). Cellular ATP pool and cell-membrane porousness were measured using Mitochondrial ToxGlo Assay (Promega Corporation, Madison, Wis.). Oxygen consumption of saponin-permeabilized cells (Hahn D, et al. Am J Physiol Cell Physiol 2019, 317(4):C665-C673) was measured by Oroboros O2k respirometry (Alleman R J, et al. Am J Physiol Heart Circ Physiol 2016, 310(10):H1360-1370; Goswami I, et al. Biophys J 2018, 114(12):2951-2964).

Restoring (rescuing) the impaired growth and respiration the mutant. Effectiveness of the potential Complex I therapeutic idebenone in restoring the ATP synthesis defects of the mutant HEK293ΔNDUFS2 was evaluated. In this procedure, mutant cells were cultured in media supplemented with 1 μM of idebenone in 96-well plates at 2,000 cells/well. Fresh media (with or without the therapeutic) were added on days 0, 3 and 5. Cellular ATP pool was assessed on day 6 of incubation by Mitochondrial ToxGlo Assay. Mutant cells were also cultured in 6-well plates at 2000 cells/well in medium supplemented 0.5, 1, 5, or 10 μM of idebenone. Fresh media containing the drug was added on days 3, 6 and 9, and the cell counts in wells of triplicates was determined following 10 days incubation. Efficacy of idebenone in improving respiration of permeabilized mutant cells was measured by O2k respirometry as described above with the following modification: subsequent to glutamate+malate injection, idebenone was injected into chambers at 1 μM final concentration. The control groups received DMSO.

Statistical analyses. Student's t-tests were performed using Microsoft Excel program (Microsoft, Redmond, Wash.) or GraphPad Prism 7 (GraphPad Software, San Diego, Calif.) to compare the means of the parent versus mutant or vehicle control versus idebenone treatment. The ECAR data were analyzed by repeated measures ANOVA using GraphPad Prism 8. Mean differences between groups were considered statistically significant at p<0.05.

Results

Construction of the recombinant mutant cell line HEK293.6NDUFS2. The 2061 bp long nucleotide sequence of Homo sapiens NDUFS2 (GenBank locus ID BC008868) was used as the template for designing sgRNA sequences using Integrated DNA Technologies' online tool (Skokie, Ill.). The 1002-bp site of the forward strand was predicted as the most reliable for mutating with 88% on-target specificity score. The guide strand predicted by this program for this site was ACGTTTGTTGAACCACATCA (SEQ ID NO:4) that is located right upstream of the protospacer adjacent motif (PAM) sequence TGG (FIG. 1A). The top and bottom sgRNA sequences for this site in 5′ to 3′ direction were CACCgACGTTTGTTGAACCACATCA (SEQ ID NO:8) and AAACTGATGTGGTTCAACAAACGTc (SEQ ID NO:9), respectively. The constructed mutant HEK293ΔNDUFS2 was found missing an approximately 49 kDa protein (FIG. 1B), corresponding to the predicted molecular weight of NDUFS2.

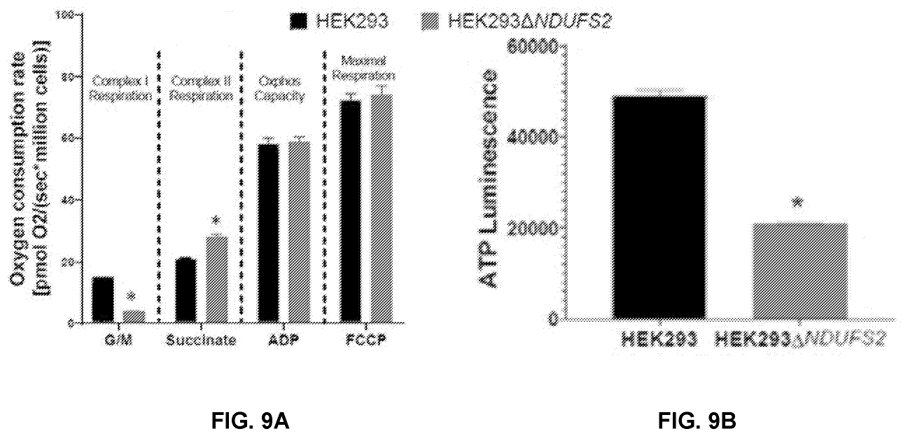

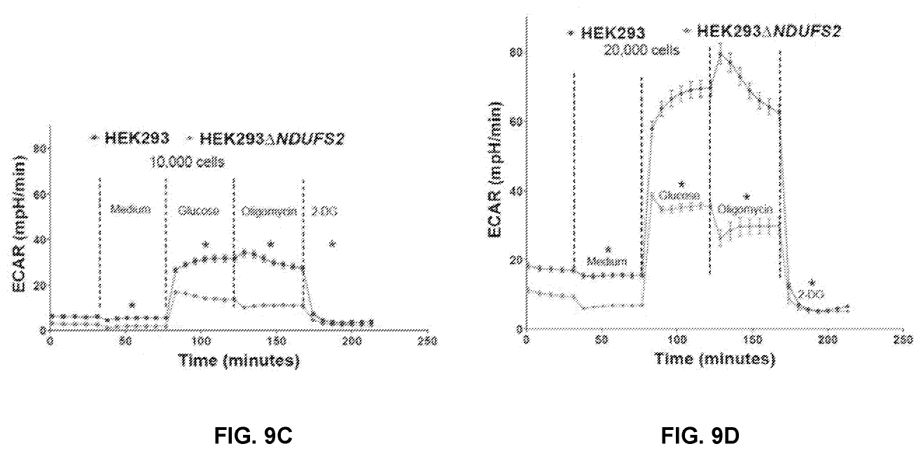

Disruption of NDUFS2 impaired oxygen consumption. Oxygen consumption rate was measured using Oroboros Oxygraph 2k. Oxygen consumption was collected at each step along ETC (FIG. 9A). Disruption of NDUFS2 decreased Complex I respiration by 75% (p<0.0001), highlighting the vitality of this subunit for Complex I respiration. Nevertheless, the Complex II respiration of the mutant increased by 40% (p=0.001), reflecting a possible compensatory regulation by the Complex II in a scenario of a functional deficiency of Complex I (FIG. 9A).

Disruption of NDUFS2 impaired glycolysis and ATP synthesis. In response to glucose injection, ECAR increased significantly in both the parent and the mutant, but the final rate of the mutant was only 50-61% lower than that of the parent (p<0.0001) (FIG. 9C & 9D). Upon treatment with oligomycin, ECAR of the parent slightly increased before starting to gradually decline, whereas that of the mutant slightly decreased and then remained stable. The final ECAR of the mutant after oligomycin treatment was 52-56% lower than that of the parent (p<0.0001). Similar trends of ECAR changes were seen when 10,000 cells (FIG. 9C) or 20,000 cells (FIG. 9D) per well were used in assays. Overall, disruption of NDUFS2 affected adversely on glycolytic capacity. Luminescence data indicated that compared to the parent, the mutant produced 57.6% less amount of ATP (p<0.0001). Impaired ATP synthesis was consistent with suppressed mitochondrial and glycolytic metabolism.

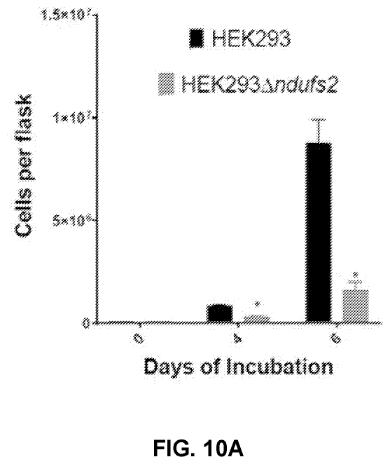

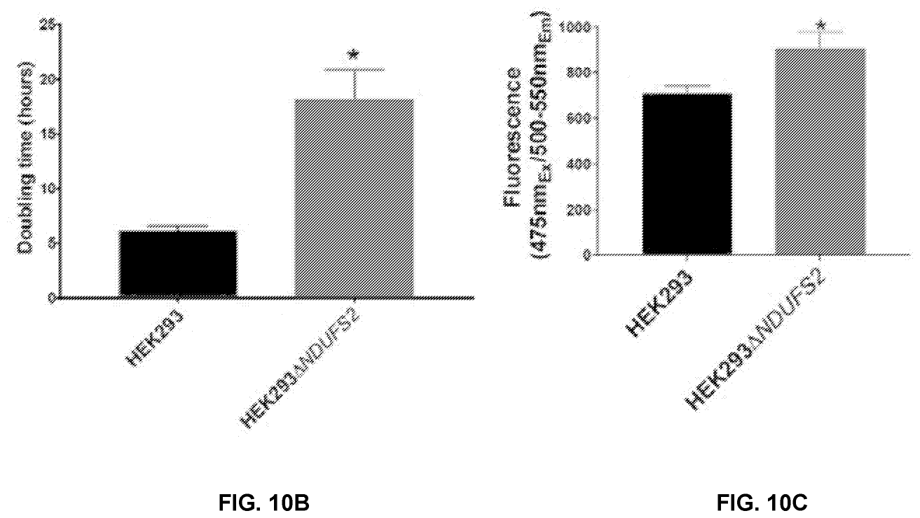

Disruption of NDUFS2 impaired growth and cell-membrane integrity. After four days of incubation, 62% fewer number of cells were recovered from the mutant compared to the parent (p=0.019). After six days of incubation, 82% fewer number of cells were recovered from the mutant (p=0.027) (FIG. 10A). The doubling time of the mutant between the days 0 and 4 was 197.4% longer than that of the parent (p=0.0477) (FIG. 10B). Cell membranes of the mutant were 27.7% more porous than those of the parent (p=0.0221) (FIG. 10C). Impaired oxygen consumption and glycolysis of the mutant may have decreased the cell proliferation capacity and also weakened the cell-membrane integrity.

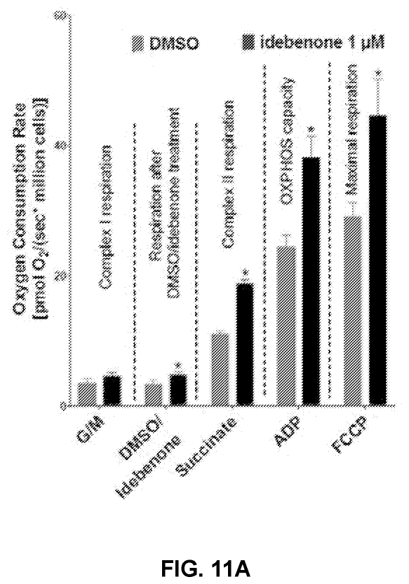

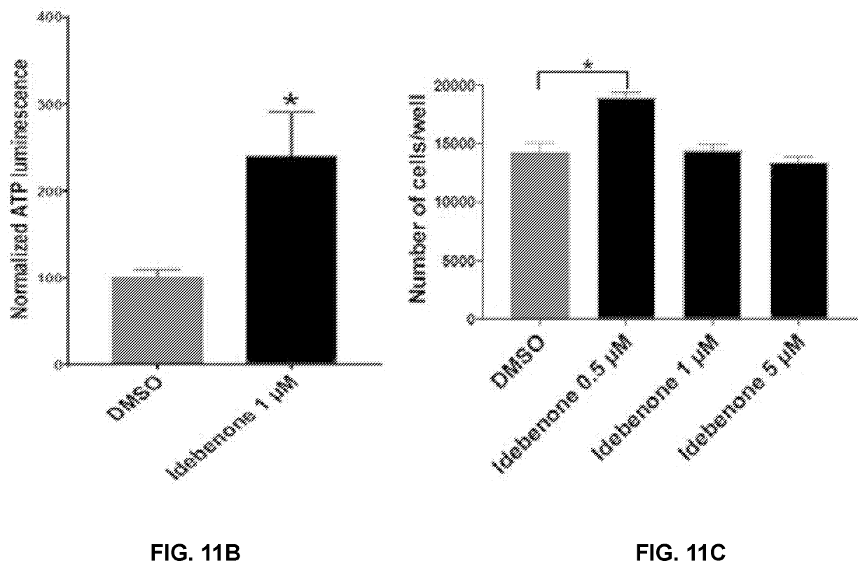

Treatment with idebenone restored altered phenotypes of the mutant. Idebenone is a short-chain benzoquinone with greater hydrophilicity (Erb M, et al. PLoS One 2012, 7(4):e36153), which has shown promise as a therapeutic for mitochondrial dysfunction (Erb M, et al. PLoS One 2012, 7(4):e36153; Haefeli R H, et al. PLoS One 2011, 6(3):e17963; Heitz F D, et al. PLoS One 2012, 7(9):e45182). One goal was to determine whether idebenone modified oxygen consumption of mutant cells by O2k respirometry. When idebenone was injected at 1 μM final concentration into the chamber carrying permeabilized mutant cells, Complex I respiration, Complex II respiration, OXPHOS capacity, and maximal respiration were improved significantly (p=0.024, 0.0001, 0.0013, and 0.0066 respectively) (FIG. 11A) Another goal was st to determine whether long-term idebenone treatment modified ATP synthesis of mutant cells by Mitochondrial ToxGlo Assay. Compared to the mutant cells treated with DMSO, those treated with 1 μM idebenone produced significantly greater amount of ATP (p=0.0101) (FIG. 11B). Treatment with 10 μM concentrations of idebenone failed to improve the ATP synthesis defects of the mutant. Another goal was to determine whether long-term idebenone treatment would influence cell growth. Due to the slow growth of mutant, cells were grown for 10 days in growth media added 0.5, 1, 5, or 10 μM idebenone. After 10 days, 32.1% more cells were recovered from cells treated with 0.5 μM idebenone compared to the DMSO control (p=0.001082) (FIG. 11C). However, treatment with 1 or 5 μM idebenone did not improve growth of the mutant (FIG. 11C), whereas treatment with 10 μM idebenone caused cell death.

### Discussion

NDUFS2 is a constituent of the metabolically important interface region of the mitochondrial Complex I (Efremov R G, et al. Nature 2010, 465(7297):441-445; Hunte C, et al. Science 2010, 329(5990):448-451). Due to the reported importance of this protein subunit to Complex I respiration and associated disease pathologies (Tuppen H A, et al. Brain 2010, 133(10):2952-2963; Haack T B, et al. J Med Genet 2012, 49(2):83-89; Haack T B, et al. J Med Genet 2012, 49(4):277-283; Loeffen J, et al. Ann Neurol 2001, 49(2):195-201; Gerber S, et al. J Med Genet 2017, 54(5):346-356; Rubrecht A, et al. Fetal Pediatr Pathol 2019:1-4; Bugiani M, et al. Biochim Biophys Acta 2004, 1659(2-3):136-147; Distelmaier, et al. Brain 2009, 132(Pt 4):833-842), a thorough examination of molecular level contribution of NDUFS2 to cellular respiration is required. Nevertheless, studies on organization and functionality on NDUFS2 have been hampered by the lack of knockout cell culture or mouse models. A defined and reliable NDUFS2 knockout cell line is essential for understanding the critical roles played by this subunit protein in OXPHOS and mitochondrial diseases.

The NDUFS2 knockout cell line was impaired in Complex I respiration. This was predicted since NDUFS2 is a subunit of Complex I and a constituent of the interfacial region of this complex. Nevertheless, quite interestingly, the mutant exhibited a greater Complex II respiration than the parent strain possibly because Complex II functions at a greater rate in order to compensate the overall oxygen consumption. ECAR of the mutant decreased significantly reflecting an impaired glycolysis as a result of NDUFS2 disruption. It is possible that decreased demand for the Complex I substrate NADH lowered the necessity of pyruvate imposing a potential negative feedback on glycolysis. The mutant also produced significantly less amounts of ATP possibly as a result of decreased respiration and glycolysis. The mutant cells proliferated slower than the parent. This was anticipated since NDUFS2 dysfunction decreased respiration and glycolysis resulting in less abundance of ATP used in cell growth. Cell-membrane integrity was seen compromised in the mutant possibly as a result of impaired metabolism.

Next investigated was whether NDUFS2 disruption could be bypassed with use of mitochondrial therapeutic. Idebenone, a short-chain benzoquinone that is more hydrophilic than ubiquinone (Erb M, et al. PLoS One 2012, 7(4):e36153), has potential as a therapeutic for conditions associated with oxidative stress and mitochondrial dysfunction (Erb M, et al. PLoS One 2012, 7(4):e36153; Haefeli R H, et al. PLoS One 2011, 6(3):e17963; Heitz F D, et al. PLoS One 2012, 7(9):e45182; Palecek T, et al. Curr Pharm Des 2015, 21(4):491-506; Strawser C J, et al. Expert Rev Neurother 2014, 14(8):949-957; Lynch D R, et al. Arch Neurol 2010, 67(8):941-947; Buyse G M, et al. Neuromuscul Disord 2011, 21(6):396-405; Buyse G M, et al. Pediatr Pulmonol 2017, 52(4):508-515; McDonald C M, et al. Neuromuscul Disord 2016, 26(8):473-480; Buyse G M, et al. Lancet 2015, 385(9979):1748-1757; Fiebiger S M, et al. J Neuroimmunol 2013, 262(1-2):66-71). Treatment with idebenone improved oxygen consumption all along the ETC, suggesting that the ubiquinone analog idebenone is able to substitute for Complex I as an electron donor to improve ETC function. Interestingly, long-term treatment with 1 μM of idebenone improved ATP pool of the mutant, whereas the long-term treatment with 0.5 μM of this therapeutic enhanced cell proliferation.

### Conclusions

Functions of NDUFS2 are vital for growth, Complex I respiration, glycolysis, ATP synthesis, cell-membrane integrity, and in vitro growth of HEK293. The respiratory effects of NDUFS2 disruption can be partially restored by treatment with the potential therapeutic idebenone. The constructed mutant is a useful and novel platform to study the efficacy of novel Complex I therapeutics.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A recombinant human cell line, wherein each cell of the recombinant human cell line has an inactivation mutation in a NADH dehydrogenase [ubiquinone] iron-sulfur protein 2 (NDUFS2) gene, wherein the human cell line does not express a functional NDUFS2 protein.

2. The recombinant human cell line of claim 1, wherein the inactivation mutation comprises a deletion or non-sense mutation in at least one coding region of the NDUFS2 gene.

3. The recombinant human cell line of claim 1, wherein the NDUFS2 gene encodes an mRNA having the nucleotide sequence SEQ ID NO:11.

4. The recombinant human cell line of claim 1, wherein the NDUFS2 gene has been mutated or deleted using CRISPR/Cas9 genome editing with a short guide RNAs (sgRNA) that targets a coding region in the NDUFS2 gene.

5. The recombinant human cell line of claim 4, wherein the sgRNA comprises the nucleic acid sequence SEQ ID NO:6, 7, 8, or 9.

6. The recombinant human cell line of claim 1, wherein the cell is a human embryonic kidney (HEK) cell.

7. The recombinant human cell line of claim 6, wherein the HEK cell is a HEK293 cell.

8. The recombinant human cell line of claim 1, wherein the cell is stably or transiently transfected with a first heterologous nucleic acid sequence.

9. The recombinant human cell line of claim 8, wherein the cell is stably or transiently transfected with a second heterologous nucleic acid sequence.

10. The recombinant human cell line of claim 1, in a culture or growth medium, or in a medium suitable for long-term storage.

11. A screening method comprising
(a) contacting the recombinant human cell line of claim 1 with a candidate agent,
(b) assaying the cell line for ATP synthesis, cell growth, complex I respiration, or a combination thereof,
wherein an increase in any one of ATP synthesis, cell growth, or complex I respiration is an indication that the candidate agent may be useful for treating a subject with a complex I defect.

12. The method of claim 11, wherein the complex I defect comprises hepatopathy, cardiomyopathy, muscle myopathies, fatal congenital lactic acidosis, Leber's hereditary optic neuropathy, or Leigh syndrome.
